(19) United States
(12) Patent Application Publication
(10) Pub. No.: US 2007/0232698 A1
(43) Pub. Date: Oct. 4, 2007

(54) Slimming Skin External Preparation and Cosmetic Containing the Same

(76) Inventors: Akira Shibuya, Kanagawa (JP); Eiko Kato, Chiba (JP); Motoaki Kamachi, Kanagawa (JP); Hirobumi Aoki, Chiba (JP)

Correspondence Address: SUGHRUE MION, PLLC, 2100 PENNSYLVANIA AVENUE, N.W., SUITE 800, WASHINGTON, DC, 20037, US

(21) Appl. No.: 11/597,884

(22) PCT Filed: May 31, 2005
(86) PCT No.: PCT/JP05/10309

§ 371 Date: Nov. 29, 2006

Related U.S. Application Data
(60) Provisional application No. 60/578,851, filed on Jun. 14, 2004.

(30) Foreign Application Priority Data
May 31, 2004 (JP) 2004-161774

(51) Int. Cl. A61K 31/195

(52) U.S. Cl. 514/561

(57) ABSTRACT

The invention provides a slimming skin external preparation and a cosmetic that contain a carnitine derivative having excellent skin affinity and percutaneous absorption properties and capable of enhancing the fat metabolism, and that can treat, reduce or prevent the obesity by functioning as a lipolytic agent to enhance the fat metabolism of general or local subcutaneous fat tissues. The slimming skin external preparation contains a particular carnitine derivative and preferably other slimming and skin-care ingredients.

### REFERENCE TO RELATED APPLICATIONS

This application is an application filed under 35 U.S.C. §111(a) claiming benefit pursuant to 35 U.S.C. §119(e) of the filing dates of Provisional Application No. 60/578,851 filed on Jun. 14, 2004 pursuant to 35 U.S.C. §111(b).

### FIELD OF THE INVENTION

The present invention relates to a slimming skin external preparation and a cosmetic containing the same. More particularly, the invention relates to a skin external preparation and a cosmetic that contain a carnitine derivative and have an effect of stimulating breakdown of subcutaneous fat.

The invention also relates to a slimming skin external preparation and a cosmetic that contain a carnitine derivative and slimming and skin-care ingredients.

### BACKGROUND OF THE INVENTION

Much of the fat in the body is the result of an accumulation of neutral fat within white adipocytes, which is caused by energy ingestion in excess of metabolism energy. Obesity means accumulation of excess fat as the body fat, and often leads to various diseases including arteriosclerosis. In addition to such health concerns, the accumulation of excess subcutaneous fat is aesthetically unfavorable. These have created a growing desire for a firm body with less body fat. However, the obesity is nowadays increasing for many reasons such as excessively rich diet, overeating, physical inactivity and stress. Thus, decreasing the subcutaneous fat and preventing the accumulation thereof are now substantial problems for people irrespective of age. Many methods have been attempted to treat the obesity, including diet restriction, exercise and external stimuli such as massage, as well as ingestion of digestion and absorption inhibiting food. However, no external preparations stimulating the fat breakdown and slimming cosmetics have been found that possess satisfactory effects of readily suppressing or reducing the subcutaneous fat which has a great influence on appearance.

The fat is metabolized into energy by processes in which a fatty acid is β-oxidized in a mitochondrion. For the fatty acid to be introduced in a mitochondrion, it must be combined with carnitine, and therefore the metabolic rate of fatty acid is dependent on the quantity of carnitine present. Accordingly, increasing the carnitine concentration in a tissue in which enhanced fatty acid metabolism is desired will be effective for fat breakdown and slimming. For this reason, percutaneous absorption of skin external preparations containing carnitine has been studied and proposed in many ways to stimulate the metabolism of subcutaneous fat (Patent Documents 1 to 4). However, such works, which employ L-carnitine and salts thereof, have been unable to achieve satisfactory slimming effects. The reason is probably that because the carnitine and salts thereof are hydrated quite easily, their direct use results in poor skin affinity and percutaneous absorption properties, and enough carnitine or the like cannot penetrate to the tissue in which the fat metabolism is to be performed.

Accordingly, there has been a need for a slimming skin external preparation that has excellent percutaneous absorption properties, can sufficiently penetrate to the tissue in which the fat metabolism is to be performed, and has a superior effect of enhancing the fat metabolism.

Meanwhile, it has been proposed that palmitoyl-L-carnitine derived from carnitine is added together with caffeine to cosmetics for slimming effects (Patent Documents 5 and 6).

Substances other than carnitine involved in fat metabolism and synthesis have been increasingly studied. Such substances under study include hydroxycitric acid and derivatives thereof known as inhibitors of fat synthetic pathway via the citric acid cycle (Nonpatent Documents 1 and 2, and Patent Document 7), substances capable of depressing functions of α-2 adrenaline that inhibits the synthesis of intracellular cAMP which acts as a second messenger in lipolysis (Nonpatent Documents 3 and 4), β-adrenergic stimulants capable of stimulating the synthesis of intracellular cAMP (Nonpatent Document 3 and Patent Document 8), and inhibitors of phosphodiesterase having effects of the decomposition of intracellular cAMP (Patent Document 9).

On the other hand, even if the fat metabolism is stimulated and the obesity is treated, excessive fat metabolism causes shortage of subcutaneous fat and sebum to result in the lack of skin vitality, dry skin and skin roughness.

Accordingly, there has been a need for a slimming skin external preparation and a cosmetic that possess a high effect of stimulating the fat metabolism and are capable of keeping the skin healthy.

[Patent Document 1] Japanese Patent No. 3434995

[Patent Document 2] JP-A-H07-309711

[Patent Document 3] JP-A-2000-16916

[Patent Document 4] JP-A-2001-64147

[Patent Document 5] French Patent No. 2694195

[Patent Document 6] Publication WO 2004/002435

[Patent Document 7] JP-A-H09-176004

[Patent Document 8] JP-A-S59-155313

[Patent Document 9] JP-A-H10-182347

[Nonpatent Document 1] Sullivan A. C. et al., “Lipids 9 (2)12” pp. 121-128, 1974

[Nonpatent Document 2] Watson Y. A. et al., “Arch. Biochem. Biophys. 135(1)” pp. 209-217, 1969

[Nonpatent Document 3] M. Lafontan and M. Berlan, “Fat cell adrenergic receptors and the control of white and brown fat cell function”, Journal of Lipid Research Vol. 34, pp. 1057-1091, 1993

[Nonpatent Document 4] Greenway F. L. et al., “Regional fat loss from the thigh in obese women after adrenergic modulation”, Clinical Therapeutics 9(6), pp. 663-669, 1987

### DISCLOSURE OF THE INVENTION

It is an object of the present invention to provide a slimming skin external preparation and a cosmetic that contain a carnitine derivative having excellent skin affinity and percutaneous absorption properties and capable of enhancing the fat metabolism, and that can treat, reduce or prevent the obesity by functioning as a lipolytic agent to enhance the fat metabolism of general or local subcutaneous fat tissues.

It is another object of the invention to provide a slimming skin external preparation and a cosmetic that contain a carnitine derivative and a slimming ingredient to achieve higher specific slimming effects.

It is a further object to provide a slimming skin external preparation and a cosmetic that possess not only high slimming effects but also good skin-moisturizing and skin-care effects.

The present inventors studied diligently in view of the previously mentioned circumstances. As a result, it has been found that: (1) particular carnitine derivatives such as acylcarnitine derivatives, alcohol carnitine ester derivatives and combinations thereof possess high skin affinity and percutaneous absorption properties, readily penetrate to the tissue in which the fat metabolism is to be performed, and favorably enhance the fat metabolism; (2) use in combination of the particular carnitine derivative and an ingredient involved in the pathway for fat metabolism and synthesis leads to an improved effect of specifically enhancing the fat metabolism; and (3) use in combination of the particular carnitine derivative and a particular skin-care ingredient provides skin-moisturizing and skin-care effects while facilitating the fat metabolism. The present invention has been completed based on the finding.

The present invention concerns the following [1] to [62]:

[1] A slimming skin external preparation comprising a carnitine derivative represented by the following formula (1): 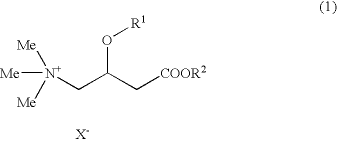
wherein R¹ is a hydrogen atom or an acyl group of 2 to 30 carbon atoms that may have a branch or an unsaturated bond, R² is a hydrogen atom or a hydrocarbon group of 1 to 22 carbon atoms that may have a branch or an unsaturated bond, R¹ and R² cannot be hydrogen atoms at the same time, Me is a methyl group, and X⁻ is an inorganic or organic anion that maintains electrical neutrality with a cation part of the carnitine derivative.

[2] The slimming skin external preparation as described in [1], wherein R¹ in the formula (1) is an acyl group of 4 to 22 carbon atoms that may have a branch or an unsaturated bond.

[3] The slimming skin external preparation as described in [1], wherein R¹ in the formula (1) is an acyl group of 14 to 22 carbon atoms that may have a branch or an unsaturated bond.

[4] The slimming skin external preparation as described in any one of [1] to [3], wherein R² in the formula (1) is a hydrogen atom or a hydrocarbon group of 1 to 18 carbon atoms that may have a branch or an unsaturated bond.

[5] The slimming skin external preparation as described in [1], wherein R¹ in the formula (1) is a hydrogen atom and R² is a hydrocarbon group of 1 to 18 carbon atoms that may have a branch or an unsaturated bond.

[6] The slimming skin external preparation as described in any one of [1] to [3], wherein R² in the formula (1) is a hydrocarbon group of 4 to 10 carbon atoms that may have a branch or an unsaturated bond.

[7] The slimming skin external preparation as described in [1], wherein R¹ in the formula (1) is an acyl group of 4 to 22 carbon atoms that may have a branch or an unsaturated bond and R² is a hydrogen atom.

[8] The slimming skin external preparation as described in any one of [1] to [7], wherein the preparation contains the carnitine derivative in an amount of 0.01 to 20% by mass.

[9] The slimming skin external preparation as described in any one of [1] to [8], wherein the preparation further contains at least one of hydroxycitric acid, a hydroxycitric acid derivative and a pharmacologically acceptable salt thereof.

[10] The slimming skin external preparation as described in [9], wherein the hydroxycitric acid is obtained from garcinia cambogia extract.

[11] The slimming skin external preparation as described in [9] or [10], wherein the hydroxycitric acid derivative is represented by the following formula (2): 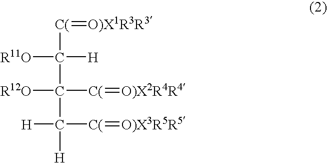
wherein R¹¹ and R¹² are each a hydrogen atom or a group detachable by biological enzyme reaction, the detachable group being represented by any of the following formula (3), R¹¹ and R¹² cannot be hydrogen atoms at the same time, X¹ to X³ are each a nitrogen atom or an oxygen atom, and R³, R⁴, R⁵, R^{3′}, R^{4′} and R^{5′} are each a hydrogen atom or a chain hydrocarbon group of 1 to 30 carbon atoms that may have a branch or an unsaturated bond (with the proviso that when X¹, X² or X³ is an oxygen atom, corresponding R^{3′}, R^{4′} or R^{5′} does not exist) 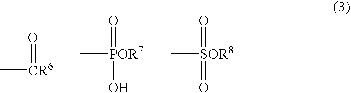
wherein R⁶ to R⁸ are each a hydrogen atom, an aryl group or a chain hydrocarbon group of 1 to 30 carbon atoms that may have a branch, an unsaturated bond or a substituent group.

[12] The slimming skin external preparation as described in [11], wherein R⁶ in the formula (3) is a chain hydrocarbon group of 7 to 23 carbon atoms that may have a branch, an unsaturated bond or a substituent group, and R⁷ and R⁸ are each a hydrogen atom or a chain hydrocarbon group of 8 to 24 carbon atoms that may have a branch, an unsaturated bond or a substituent group.

[13] The slimming skin external preparation as described in [11], wherein R³ to R⁵ in the formula (2) are each a hydrogen atom or a chain hydrocarbon group of 1 to 30 carbon atoms that may have a branch or an unsaturated bond, and X¹ to X³ are all oxygen atoms, and wherein R⁶ in the formula (3) is a chain hydrocarbon group of 7 to 23 carbon atoms that may have a branch, an unsaturated bond or a substituent group, and R⁷ and R⁸ are each a hydrogen atom or a chain hydrocarbon group of 8 to 24 carbon atoms that may have a branch, an unsaturated bond or a substituent group.

[14] The slimming skin external preparation as described in [11], wherein R¹² in the formula (2) is a hydrogen atom, R³ to R⁵ are all hydrogen atoms, and X¹ to X³ are all oxygen atoms, and wherein R⁶ in the formula (3) is a chain hydrocarbon group of 13 to 21 carbon atoms that may have a branch, an unsaturated bond or a substituent group.

[15] The slimming skin external preparation as described in [11], wherein R¹² in the formula (2) is a hydrogen atom, R³ to R⁵ are each a hydrogen atom or a chain hydrocarbon group of 1 to 30 carbon atoms that may have a branch or an unsaturated bond, R³ to R⁵ cannot be hydrogen atoms at the same time, and X¹ to X³ are all oxygen atoms, and wherein R⁶ in the formula (3) is a chain hydrocarbon group of 13 to 21 carbon atoms that may have a branch, an unsaturated bond or a substituent group.

[16] The slimming skin external preparation as described in any one of [9] to [15], wherein the preparation contains at least one of the hydroxycitric acid, the hydroxycitric acid derivative and the pharmacologically acceptable salt thereof in an amount of 0.01 to 20% by mass.

[17] The slimming skin external preparation as described in any one of [1] to [8], wherein the preparation further contains an α-2 adrenergic inhibitor.

[18] The slimming skin external preparation as described in [17], wherein the α-2 adrenergic inhibitor is at least one of yohimbine, phentolamine, phenoxybenzamine, tolazoline, ergotamine, ergotoxine, dihydroergotamine, ergometrine, methylergometrine, dihydroergotoxine, rauwolscine, piperoxan, derivatives thereof and pharmacologically acceptable salts thereof.

[19] The slimming skin external preparation as described in [17], wherein the α-2 adrenergic inhibitor is obtained from a plant extract.

[20] The slimming skin external preparation as described in [17], wherein the α-2 adrenergic inhibitor is obtained from at least one of a ginkgo extract, a hedera rhombea extract and a chestnut extract.

[21] The slimming skin external preparation as described in any one of [17] to [20], wherein the preparation contains the α-2 adrenergic inhibitor in an amount of 0.0001 to 10% by mass.

[22] The slimming skin external preparation as described in any one of [1] to [8], wherein the preparation further contains a β-adrenergic stimulant.

[23] The slimming skin external preparation as described in [22], wherein the β-adrenergic stimulant is at least one of isoproterenol, epinephrine, norepinephrine, dobutamine, dopamine, butopamine, salbutamol, terbutaline, isoetharine, protokylol, fenoterol, metaproterenol, clorprenaline, hexoprenaline, trimetoquinol, procaterol hydrochloride, prenalterol, forskolin, disodium(R,R)-5-[2-[[2-(3-chlorophenyl)-2-hydroxyethyl]-amino]propyl]-1,3-benzodioxol-2,2-dicarboxylate, (R,R)-4-[2-({2-[(3-chlorophenyl)-2-hydroxyethyl]amino}propyl)phenyl]phenoxyacetic acid, {2-hydroxy-5-[2-({2-hydroxy-3-[4-(1-methyl-4-trifluoromethyl)-1H-imidazol-2-yl]phenoxy}propyl)amino]ethoxy}-benzamide monomethane sulfonate, erythro-DL-1-(7-methylindane-4-yloxy)-3-isopropylaminobutane-2-ol, derivatives thereof and pharmacologically acceptable salts thereof.

[24] The slimming skin external preparation as described in [22], wherein the β-adrenergic stimulant is obtained from a plant extract.

[25] The slimming skin external preparation as described in [22], wherein the β-adrenergic stimulant is obtained from at least one of a coleus forskohlii extract (forskolin), an ipomoea hederacea extract, an ipomoea batata extract, a salvia officinalis extract, a salvia miltiorrhiza extract and a rosmarinus officinalis (rosemary) extract.

[26] The slimming skin external preparation as described in any one of [22] to [25], wherein the preparation contains the β-adrenergic stimulant in an amount of 0.0001 to 10% by mass.

[27] The slimming skin external preparation as described in any one of [1] to [8], wherein the preparation further contains an inhibitor of phosphodiesterase.

[28] The slimming skin external preparation as described in [27], wherein the inhibitor of phosphodiesterase is at least one of theophylline, theobromine, aminophylline, xanthine, isobutylmethylxanthine, apigenin, amentoflavone, bilobetin, sciadopiticin, ginkgonetin, derivatives thereof and pharmacologically acceptable salts thereof.

[29] The slimming skin external preparation as described in [27], wherein the inhibitor of phosphodiesterase is obtained from a plant extract.

[30] The slimming skin external preparation as described in [27], wherein the inhibitor of phosphodiesterase is obtained from at least one of a tea extract, a coffee extract, a guarana extract, a yerba mate extract, a cola extract, a ginkgo extract, a sequoia sempervirens extract, a yew extract and a selaginella shakotanensis extract.

[31] The slimming skin external preparation as described in any one of [27] to [30], wherein the preparation contains the inhibitor of phosphodiesterase in an amount of 0.0001 to 10% by mass.

[32] The slimming skin external preparation as described in any one of [1] to [8], wherein the preparation further contains a saturated or unsaturated fatty acid of 12 to 22 carbon atoms that may have a branch, and/or a pharmacologically acceptable salt thereof.

[33] The slimming skin external preparation as described in [32], wherein the fatty acid is at least one of lauric acid, myristic acid, palmitic acid, stearic acid, behenic acid, oleic acid, isostearic acid, 12-hydroxystearic acid, undecylenic acid and hexyldecanoic acid.

[34] The slimming skin external preparation as described in [32], wherein the fatty acid is a coconut oil fatty acid.

[35] The slimming skin external preparation as described in any one of [32] to [34], wherein the preparation contains the saturated or unsaturated fatty acid of 12 to 22 carbon atoms that may have a branch, and/or the pharmacologically acceptable salt thereof in an amount of 0.001 to 20% by mass.

[36] The slimming skin external preparation as described in any one of [1] to [8], wherein the preparation further contains at least one of ascorbic acid, an ascorbic acid derivative, and a pharmacologically acceptable salt thereof.

[37] The slimming skin external preparation as described in [36], wherein the ascorbic acid is L-ascorbic acid.

[38] The slimming skin external preparation as described in [36] or [37], wherein the pharmacologically acceptable salt of the ascorbic acid is sodium L-ascorbate and/or magnesium L-ascorbate.

[39] The slimming skin external preparation as described in any one of [36] to [38], wherein the ascorbic acid derivative is at least one of L-ascorbyl stearate, L-ascorbyl palmitate, L-ascorbyl dipalmitate, L-ascorbyl tetraisopalmitate and L-ascorbic acid-2-glucoside.

[40] The slimming skin external preparation as described in any one of [36] to [38], wherein the pharmacologically acceptable salt of the ascorbic acid derivative is disodium L-ascorbate sulfate.

[41] The slimming skin external preparation as described in any one of [36] to [38], wherein the ascorbic acid derivative is ascorbic acid-2-phosphate and/or a higher fatty acid ester thereof, represented by the following formula (4): 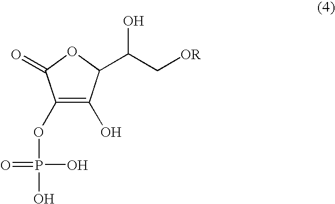
wherein R is a hydrogen atom or an acyl residue of the higher fatty acid.

[42] The slimming skin external preparation as described in [41], wherein R in the formula (4) is an acyl residue of lauric acid, myristic acid, palmitic acid, stearic acid or 2-hexyldecanoic acid.

[43] The slimming skin external preparation as described in any one of [36] to [38], [41] and [42], wherein the pharmacologically acceptable salt of the ascorbic acid derivative is a sodium salt, a potassium salt, a magnesium salt or a zinc salt of ascorbic acid-2-phosphate and/or a higher fatty acid ester thereof, represented by the following formula (4): 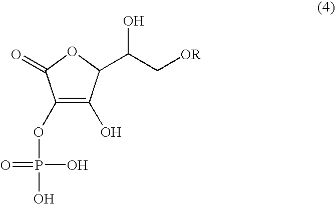
wherein R is a hydrogen atom or an acyl residue of the higher fatty acid.

[44] The slimming skin external preparation as described in [43], wherein R in the formula (4) is an acyl residue of lauric acid, myristic acid, palmitic acid, stearic acid or 2-hexyldecanoic acid.

[45] The slimming skin external preparation as described in any one of [36] to [44], wherein the preparation contains at least one of the ascorbic acid, the ascorbic acid derivative and the pharmacologically acceptable salt thereof in an amount of 0.01 to 20% by mass.

[46] The slimming skin external preparation as described in any one of [1] to [8], wherein the preparation further contains at least one of tocopherol, a tocopherol derivative and a pharmacologically acceptable salt thereof.

[47] The slimming skin external preparation as described in [46], wherein the tocopherol is at least one of α-tocopherol, β-tocopherol, γ-tocopherol and δ-tocopherol.

[48] The slimming skin external preparation as described in [46] or [47], wherein the tocopherol derivative is at least one of an α-tocopherol derivative, a β-tocopherol derivative, a γ-tocopherol derivative and a δ-tocopherol derivative.

[49] The slimming skin external preparation as described in [48], wherein the tocopherol derivative is tocopherol phosphate.

[50] The slimming skin external preparation as described in [49], wherein the tocopherol phosphate is a compound represented by the following formula (5): 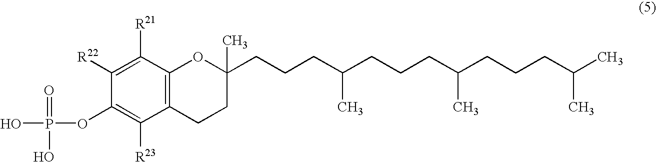
wherein R²¹, R²² and R²³ are each a hydrogen atom or a methyl group.

[51] The slimming skin external preparation as described in [48], wherein the tocopherol derivative is a tocopherol aminoalkylcarboxylate.

[52] The slimming skin external preparation as described in [51], wherein the tocopherol aminoalkylcarboxylate is a compound represented by the following formula (6): 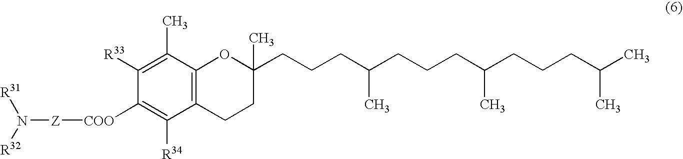
wherein R³¹ and R³² are the same or different lower alkyl groups or each a hydrogen atom, R³³ and R³⁴ are each a hydrogen atom or a methyl group, and Z is a branched or linear alkylene group that may have a substituent group.

[53] The slimming skin external preparation as described in [51] or [52], wherein the aminoalkylcarboxylic acid of the tocopherol aminoalkylcarboxylate is a compound selected from glycine, alanine, β-alanine, valine, leucine, isoleucine, phenylalanine, methionine, cysteine, serine, threonine, tyrosine, thyroxine, histidine, proline, 4-hydroxyproline, aspartic acid, glutamic acid, N-alkyl derivatives thereof and N,N-dialkyl derivatives thereof.

[54] The slimming skin external preparation as described in any one of [46] to [53], wherein the preparation contains at least one of the tocopherol, the tocopherol derivative and the pharmacologically acceptable salt thereof in an amount of 0.01 to 10% by mass.

[55] A cosmetic comprising the slimming skin external preparation of any one of [1] to [8].

[56] A cosmetic comprising the slimming skin external preparation of any one of [9] to [16].

[57] A cosmetic comprising the slimming skin external preparation of any one of [17] to [21].

[58] A cosmetic comprising the slimming skin external preparation of any one of [22] to [26].

[59] A cosmetic comprising the slimming skin external preparation of any one of [27] to [31].

[60] A cosmetic comprising the slimming skin external preparation of any one of [32] to [35].

[61] A cosmetic comprising the slimming skin external preparation of any one of [36] to [45].

[62] A cosmetic comprising the slimming skin external preparation of any one of [46] to [54].

The slimming skin external preparation and cosmetic according to the present invention contain the particular carnitine derivative that is superior to L-carnitine and salts thereof in skin affinity and percutaneous absorption properties and has a sufficient effect of enhancing the fat metabolism. The particular carnitine derivative being an active ingredient can readily penetrate to the subcutaneous fat tissue to be metabolized. Accordingly, the slimming skin external preparation and cosmetic of the invention can favorably function as a lipolytic agent that enhances the fat metabolism of general or local subcutaneous fat tissues, and thus can treat, suppress or prevent the obesity in desired areas.

The slimming skin external preparation and cosmetic that contain the particular carnitine derivative and slimming ingredient attain various functions to provide improved effects of stimulating the fat metabolism and achieve higher specific slimming effects.

The slimming skin external preparation and cosmetic that contain the particular carnitine derivative and skin-care ingredient possess not only high slimming effects brought about by the carnitine derivative but also good skin-moisturizing and skin-care effects to keep the skin healthy.

### Preferred Embodiments of the Invention

The present invention will be described in detail hereinbelow.

The slimming skin external preparation and cosmetic according to the present invention contain a particular carnitine derivative.

Carnitine Derivative

The carnitine derivative used in the invention is a compound represented by the formula (1):

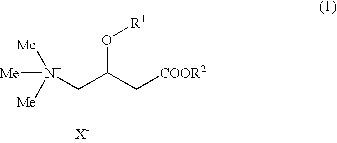

In the formula (1), R¹ is a hydrogen atom or an acyl group of 2 to 30 carbon atoms that may have a branch or an unsaturated bond, R² is a hydrogen atom or a hydrocarbon group of 1 to 22 carbon atoms that may have a branch or an unsaturated bond, R¹ and R² cannot be hydrogen atoms at the same time, Me is a methyl group, and X⁻ is an inorganic or organic anion that maintains electrical neutrality with a cation part of the carnitine derivative.

Examples of the carnitine derivatives include acylcarnitine derivatives, alcohol carnitine ester derivatives, and combinations thereof.

The acylcarnitine derivatives employable as the carnitine derivatives in the invention are compounds of the formula (1) in which R¹ is an acyl group of 2 to 30 carbon atoms that may have a branch or an unsaturated bond, and R² is a hydrogen atom. More specifically, the acylcarnitine derivatives employable in the invention are compounds in which R¹ is an acyl group of 2 to 30, preferably 4 to 22, and more preferably 14 to 22 carbon atoms that may have a branch or an unsaturated bond in the carbon chain, and R² is a hydrogen atom.

Specific examples of the acylcarnitine derivatives include compounds of the formula (1) in which R¹ is an acyl group such as hexanoyl, 2-methylpentanoyl, 3-methylpentanoyl, 4-methylpentanoyl, 2-ethylbutanoyl, heptanoyl, 2-methylhexanoyl, 3-methylhexanoyl, 4-methylhexanoyl, 2-ethylpentanoyl, 3-ethylpentanoyl, octanoyl, 2-methylheptanoyl, 3-methylheptanoyl, 4-methylheptanoyl, 5-methylheptanoyl, 6-methylheptanoyl, 2-ethylhexanoyl, 3-ethylhexanoyl, 4-ethylhexanoyl, 2-propylpentanoyl, nonanoyl, decanoyl, undecanoyl, 10-undecenoyl, dodecanoyl, tridecanoyl, tetradecanoyl, pentadecanoyl, hexadecanoyl, 9-hexadecenoyl, heptadecanoyl, octadecanoyl, isostearyl, cis-9-octadecenoyl, 11-octadecenoyl, cis,cis-9,12-octadecadienoyl, 9,12,15-octadecatrienoyl, 6,9,12-octadecatrienoyl, 9,11,13-octadecatrienoyl, nonadecanoyl, 2,6,10,14-tetramethylpentadecanoyl, icosanoyl, 8,11-icosadienoyl, 5,8,11-icosatrienoyl, 5,8,11,14-icosatetraenoyl, 3,7,11,15-tetramethylhexadecanoyl, heneicosanoyl or docosanoyl group, and R² is a hydrogen atom.

The alcohol carnitine ester derivatives employable as the carnitine derivatives in the invention are compounds of the formula (1) in which R¹ is a hydrogen atom and R² is a hydrocarbon group of 1 to 22 carbon atoms that may have a branch or an unsaturated bond. More specifically, the alcohol carnitine ester derivatives employable in the invention are compounds in which R¹ is a hydrogen atom and R² is a hydrocarbon group of 1 to 22, and preferably 1 to 18 carbon atoms that may have a branch or an unsaturated bond in the carbon chain.

Specific examples of the alcohol carnitine ester derivatives include compounds of the formula (1) in which R¹ is a hydrogen atom and R² is a hydrocarbon group such as methyl, ethyl, propyl, isopropyl, butyl, 1-methylpropyl, 2-methylpropyl, pentyl, 1-methylbutyl, 2-methylbutyl, 3-methylbutyl, 1-ethylpropyl, hexyl, 1-methylpentyl, 2-methylpentyl, 3-methylpentyl, 4-methylpentyl, 1-ethylbutyl, 2-ethylbutyl, heptyl, 1-methylhexyl, 2-methylhexyl, 3-methylhexyl, 4-methylhexyl, 5-methylhexyl, 1-ethylpentyl, 2-ethylpentyl, 3-ethylpentyl, octyl, 1-methylheptyl, 2-methylheptyl, 3-methylheptyl, 4-methylheptyl, 5-methylheptyl, 6-methylheptyl, 1-ethylhexyl, 2-ethylhexyl, 3-ethylhexyl, 4-ethylhexyl, 1-propylpentyl, 2-propylpentyl, nonyl, decyl, undecyl, 10-undecenyl, dodecyl, tridecyl, tetradecyl, pentadecyl, hexadecyl, 9-hexadecenyl, heptadecyl, octadecyl, isostearyl, cis-9-octadecenyl, 11-octadecenyl, cis,cis-9,12-octadecadienyl, 9,12,15-octadecatrienyl, 6,9,12-octadecatrienyl, 9,11,13-octadecatrienyl, nonadecyl, 2,6,10,14-tetramethylpentadecyl, icosanyl, 8,11-icosadienyl, 5,8,11-icosatrienyl, 5,8,11,14-icosatetraenyl, 3,7,11,15-tetramethylhexadecyl, heneicosanyl or docosanyl group.

The carnitine derivatives may be combinations of the aforesaid acylcarnitine derivatives and alcohol carnitine ester derivatives. That is, the carnitine derivatives may be such that R¹ and R² in the formula (1) are substituent groups other than the hydrogen atom. Such carnitine derivatives are compounds of the formula (1) in which R¹ is an acyl group of 2 to 30 carbon atoms that may have a branch or an unsaturated bond and R² is a hydrocarbon group of 1 to 22 carbon atoms that may have a branch or an unsaturated bond.

The carnitine derivatives of the formula (1) in which R¹ and R² are groups other than the hydrogen atom include compounds in which R¹ is an acyl group of 2 to 30 carbon atoms and R² is a hydrocarbon group of 1 to 22 carbon atoms, preferably in which R¹ is an acyl group of 4 to 22 carbon atoms and R² is a hydrocarbon group of 1 to 18 carbon atoms, and more preferably in which R¹ is an acyl group of 14 to 22 carbon atoms and R² is a hydrocarbon group of 4 to 10 carbon atoms. The groups R¹ and R² may each have a branch or an unsaturated bond in the carbon chain.

Specific examples of such carnitine derivatives include compounds of the formula (1) in which R¹ is an acyl group such as acetyl, propionoyl, butanoyl, pentanoyl, hexanoyl, 2-methylpentanoyl, 3-methylpentanoyl, 4-methylpentanoyl, 2-ethylbutanoyl, heptanoyl, 2-methylhexanoyl, 3-methylhexanoyl, 4-methylhexanoyl, 2-ethylpentanoyl, 3-ethylpentanoyl, octanoyl, 2-methylheptanoyl, 3-methylheptanoyl, 4-methylheptanoyl, 5-methylheptanoyl, 6-methylheptanoyl, 2-ethylhexanoyl, 3-ethylhexanoyl, 4-ethylhexanoyl, 2-propylpentanoyl, nonanoyl, decanoyl, undecanoyl, 10-undecenoyl, dodecanoyl, tridecanoyl, tetradecanoyl, pentadecanoyl, hexadecanoyl, 9-hexadecenoyl, heptadecanoyl, octadecanoyl, isostearyl, cis-9-octadecenoyl, 11-octadecenoyl, cis,cis-9,12-octadecadienoyl, 9,12,15-octadecatrienoyl, 6,9,12-octadecatrienoyl, 9,11,13-octadecatrienoyl, nonadecanoyl, 2,6,10,14-tetramethylpentadecanoyl, icosanoyl, 8,11-icosadienoyl, 5,8,11-icosatrienoyl, 5,8,11,14-icosatetraenoyl, 3,7,11,15-tetramethylhexadecanoyl, heneicosanoyl or docosanoyl group, and R² is a hydrocarbon group such as methyl, ethyl, propyl, isopropyl, butyl, 1-methylpropyl, 2-methylpropyl, pentyl, 1-methylbutyl, 2-methylbutyl, 3-methylbutyl, 1-ethylpropyl, hexyl, 1-methylpentyl, 2-methylpentyl, 3-methylpentyl, 4-methylpentyl, 1-ethylbutyl, 2-ethylbutyl, heptyl, 1-methylhexyl, 2-methylhexyl, 3-methylhexyl, 4-methylhexyl, 5-methylhexyl, 1-ethylpentyl, 2-ethylpentyl, 3-ethylpentyl, octyl, 1-methylheptyl, 2-methylheptyl, 3-methylheptyl, 4-methylheptyl, 5-methylheptyl, 6-methylheptyl, 1-ethylhexyl, 2-ethylhexyl, 3-ethylhexyl, 4-ethylhexyl, 1-propylpentyl, 2-propylpentyl, nonyl, decyl, undecyl, 10-undecenyl, dodecyl, tridecyl, tetradecyl, pentadecyl, hexadecyl, 9-hexadecenyl, heptadecyl, octadecyl, isostearyl, cis-9-octadecenyl, 11-octadecenyl, cis,cis-9,12-octadecadienyl, 9,12,15-octadecatrienyl, 6,9,12-octadecatrienyl, 9,11,13-octadecatrienyl, nonadecyl, 2,6,10,14-tetramethylpentadecyl, icosanyl, 8,11-icosadienyl, 5,8,11-icosatrienyl, 5,8,11,14-icosatetraenyl, 3,7,11,15-tetramethylhexadecyl, heneicosanyl or docosanyl group.

In the carnitine derivatives of the above formula (1), X⁻ is an inorganic or organic anion that maintains electrical neutrality with a cation part of the carnitine derivative. The inorganic or organic anions X⁻ that maintain electrical neutrality with a cation of the carnitine derivative include inorganic anions such as chloride ion, nitrate ion, sulfate ion, carbonate ion and hydrogen carbonate ion; and organic anions obtained from ionization of organic acid compounds such as formic acid, acetic acid, glycine, oxalic acid, tartaric acid and fumaric acid.

The carnitine derivatives for use in the present invention may be produced from commercially available L-carnitines as starting materials. For example, the acylcarnitine derivatives may be manufactured by a method described in Analyst (1990, 115(5), 511-516), and the alcohol carnitine ester derivatives by a method disclosed in Journal of Organic Chemistry (1995, 60(25), 8318-8319). Appropriate combination of these methods permits production of the carnitine derivatives having the two functional groups: acyl and ester groups, namely, the carnitine derivatives of the formula (1) in which R¹ and R² are groups other than the hydrogen atom. A method described in JP-A-H08-295616 also is capable of manufacturing such combined carnitine derivatives. These methods allow the starting carnitine to be in the form of any of internal salts, inorganic salts such as hydrochloride and sodium salt, and organic salts such as oxalate, tartrate and fumarate.

Slimming Skin External Preparation and Cosmetic

The slimming skin external preparation and cosmetic of the invention contain the above-described particular carnitine derivative.

In the slimming skin external preparation, the carnitine derivative content is generally in the range of 0.01 to 20% by mass, and preferably 0.05 to 12% by mass of the skin external preparation. When the carnitine derivative has this amount, the skin external preparation can quickly penetrate into the skin and can provide expected effects and efficacies. In the cosmetic of the invention as well, the carnitine derivative content is desirably similar to that in the slimming skin external preparation, although not particularly limited thereto.

In addition to the carnitine derivative, the slimming skin external preparation and cosmetic of the invention may contain other slimming and skin-care ingredients.

Slimming Ingredients

The carnitine derivative probably functions to increase the in-tissue concentration of the carnitine which acts as a mediator when the fatty acid is incorporated into mitochondrion. Accordingly, combined use of the carnitine derivative and a slimming ingredient involved in various stages of the pathway for fat metabolism or synthesis other than the fatty acid incorporation into mitochondrion, will lead to synergistically high slimming effects.

(Fat Synthetic Pathway Inhibitor)

Such slimming ingredients include hydroxycitric acid known as inhibitors of fat synthetic pathway via the citric acid cycle, hydroxycitric acid derivatives and pharmacologically acceptable salts thereof. The invention may employ one or more such slimming ingredients. These ingredients are expected to inhibit fat accumulation.

The hydroxycitric acid is found in great abundance in plant extracts such as garcinia cambogia. The invention may employ hydroxycitric acid obtained from plant extracts or synthesized by chemical processes.

Hydroxycitric acid derivatives having the formula (2) are preferable in terms of excellent percutaneous absorption properties: 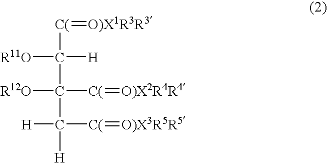
wherein R¹¹ and R¹² are each a hydrogen atom or a group detachable by biological enzyme reaction, and R¹¹ and R¹² cannot be hydrogen atoms at the same time.

The detachable group is represented by any of the following formula (3):

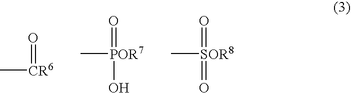

Specifically, at least one of R¹¹ and R¹² is preferably the detachable group of the formula (3). Either or both R¹¹ and R¹² may be such groups. In a preferred embodiment, either R¹¹ or R¹² is the detachable group. In a particularly preferred embodiment, R¹¹ is the detachable group of the formula (3) and R¹² is a hydrogen atom.

In the formula (3), R⁶ to R⁸ are each a hydrogen atom, an aryl group or a chain hydrocarbon group of 1 to 30 carbon atoms that may have a branch, an unsaturated bond or a substituent group. As used herein, the group detachable by biological enzyme reaction means a group that is hydrolyzed by a hydrolase such as esterase present in the living body with the result that R¹¹ and R¹² are both hydrogen atoms.

The aryl groups include phenyl, naphthyl, furyl, thienyl and pyridyl groups. The chain hydrocarbon groups of 1 to 30 carbon atoms that may have a branch, an unsaturated bond or a substituent group, include those that constitute part of the acyl group described later as R¹¹ and/or R¹².

Preferably, R⁶ to R⁸ are each a hydrogen atom or a chain hydrocarbon group of 1 to 30 carbon atoms that may have a branch, an unsaturated bond or a substituent group.

More specifically, R⁶ is a chain hydrocarbon group of 7 to 23, preferably 13 to 21 carbon atoms that may have a branch, an unsaturated bond or a substituent group; more preferably, it is an alkyl group of 13 to 21 carbon atoms that may have a branch. R⁷ and R⁸ are each a hydrogen atom or a chain hydrocarbon group of 8 to 24, preferably 14 to 22 carbon atoms that may have a branch, an unsaturated bond or a substituent group; more preferably, they are hydrogen atoms.

Desirably, the groups of the formula (3) detachable by biological enzyme reaction have 8 to 24 carbon atoms, and preferably 14 to 22 carbon atoms.

The substituent groups include halogen atoms, amino groups, cyano groups, alkoxy groups and nitro groups.

Specific examples of the hydroxycitric acid derivatives in which the hydroxyl group in the molecule is modified include compounds of the formula (2) in which R¹¹ and/or R¹² is a group selected from hexanoyl, 2-methylpentanoyl, 3-methylpentanoyl, 4-methylpentanoyl, 2-ethylbutanoyl, heptanoyl, 2-methylhexanoyl, 3-methylhexanoyl, 4-methylhexanoyl, 2-ethylpentanoyl, 3-ethylpentanoyl, octanoyl, 2-methylheptanoyl, 3-methylheptanoyl, 4-methylheptanoyl, 5-methylheptanoyl, 6-methylheptanoyl, 2-ethylhexanoyl, 3-ethylhexanoyl, 4-ethylhexanoyl, 2-propylpentanoyl, nonanoyl, decanoyl, undecanoyl, 10-undecenoyl, dodecanoyl, tridecanoyl, tetradecanoyl, pentadecanoyl, hexadecanoyl, 9-hexadecenoyl, heptadecanoyl, octadecanoyl, isostearyl, cis-9-octadecenoyl, 11-octadecenoyl, cis,cis-9,12-octadecadienoyl, 9,12,15-octadecatrienoyl, 6,9,12-octadecatrienoyl, 9,11,13-octadecatrienoyl, nonadecanoyl, 2,6,10,14-tetramethylpentadecanoyl, icosanoyl, 8,11-icosadienoyl, 5,8,11-icosatrienoyl, 5,8,11,14-icosatetraenoyl, 3,7,11,15-tetramethylhexadecanoyl, heneicosanoyl and docosanoyl groups.

Of these, preferable are compounds of the formula (2) in which R¹¹ and/or R¹² is a group selected from octanoyl, decanoyl, undecanoyl, dodecanoyl, hexadecanoyl, octadecanoyl and isostearyl groups.

More preferred are compounds in which R¹¹ is a group selected from octanoyl, decanoyl, undecanoyl, dodecanoyl, hexadecanoyl, octadecanoyl and isostearyl groups, and R¹² is a hydrogen atom.

In the formula (2), X¹ to X³ are each a nitrogen atom or an oxygen atom, R³, R⁴, R⁵, R^{3′}, R^{4′} and R^{5′} are each a hydrogen atom or a chain hydrocarbon group of 1 to 30 carbon atoms that may have a branch or an unsaturated bond (with the proviso that when X¹, X² or X³ is an oxygen atom, corresponding R^{3′}, R^{4′} or R^{5′} does not exist).

When any of X¹ to X³ is a nitrogen atom, the group —CONR^{m}R^{m′} (m and m′ are identical numbers of 3, 4 or 5 corresponding to X¹ to X³) is a substituted or unsubstituted amide group that is decomposable by biological enzyme reaction. When any of X¹ to X³ is an oxygen atom, the group —COOR^{m} (m is a number of 3, 4 or 5 corresponding to X¹ to X³) is a carboxyl group, or an ester group decomposable by biological enzyme reaction.

As used herein, the substituted or unsubstituted amide group that is decomposable by biological enzyme reaction means a substituted or unsubstituted amide group that is hydrolyzed by a hydrolase such as amidase present in the living body with the result that it is converted into a carboxyl group.

As used herein, the ester group decomposable by biological enzyme reaction means an ester group that is hydrolyzed by a hydrolase such as esterase present in the living body with the result that it is converted into a carboxyl group.

With the proviso that the above condition is satisfied, R³, R⁴, R⁵, R^{3′}, R^{4′} and R^{5′} are each a hydrogen atom or a chain hydrocarbon group of 1 to 30, preferable 8 to 24, and more preferably 14 to 22 carbon atoms that may have a branch or an unsaturated bond; more preferably they are each a hydrogen atom or an alkyl group of 14 to 22 carbon atoms that may have a branch. Furthermore, they may be saccharide residues derived from monosaccharides and polysaccharides.

Specific examples of R³, R⁴, R⁵, R^{3′}, R^{4′} and R^{5′} include a hydrogen atom and methyl, ethyl, propyl, isopropyl, butyl, 1-methylpropyl, 2-methylpropyl, pentyl, 1-methylbutyl, 2-methylbutyl, 3-methylbutyl, 1-ethylpropyl, hexyl, 1-methylpentyl, 2-methylpentyl, 3-methylpentyl, 4-methylpentyl, 1-ethylbutyl, 2-ethylbutyl, heptyl, 1-methylhexyl, 2-methylhexyl, 3-methylhexyl, 4-methylhexyl, 5-methylhexyl, 1-ethylpentyl, 2-ethylpentyl, 3-ethylpentyl, octyl, 1-methylheptyl, 2-methylheptyl, 3-methylheptyl, 4-methylheptyl, 5-methylheptyl, 6-methylheptyl, 1-ethylhexyl, 2-ethylhexyl, 3-ethylhexyl, 4-ethylhexyl, 1-propylpentyl, 2-propylpentyl, nonyl, decyl, undecyl, 10-undecenyl, dodecyl, tridecyl, tetradecyl, pentadecyl, hexadecyl, 9-hexadecenyl, heptadecyl, octadecyl, isostearyl, cis-9-octadecenyl, 11-octadecenyl, cis,cis-9,12-octadecadienyl, 9,12,15-octadecatrienyl, 6,9,12-octadecatrienyl, 9,11,13-octadecatrienyl, nonadecyl, 2,6,10,14-tetramethylpentadecyl, icosanyl, 8,11-icosadienyl, 5,8,11-icosatrienyl, 5,8,11,14-icosatetraenyl, 3,7,11,15-tetramethylhexadecyl, heneicosanyl and docosanyl groups.

With the proviso that the above condition is satisfied, R³, R⁴, R⁵, R^{3′}, R^{4′} and R^{5′} in a preferred embodiment are each a hydrogen atom or a group selected from methyl, ethyl, propyl, isopropyl, butyl, hexyl, octyl, decyl, undecyl, dodecyl, tridecyl, tetradecyl, pentadecyl, hexadecyl, heptadecyl, octadecyl and isostearyl groups.

In a more preferred embodiment, one or two of R³, R⁴, R⁵, R^{3′}, R^{4′} and R^{5′} are groups selected from methyl, ethyl, propyl, isopropyl, butyl, hexyl, octyl, decyl, undecyl, dodecyl, tridecyl, tetradecyl, pentadecyl, hexadecyl, heptadecyl, octadecyl and isostearyl groups, and the other groups are hydrogen atoms.

X¹ to X³ are each a nitrogen atom or an oxygen atom and may be the same or different from each other. Preferably, they are the same atoms, and more preferably oxygen atoms.

When X¹ to X³ are all oxygen atoms, the modified carboxyl group sites are —COOR^{m} (m is a number of 3, 4 or 5 corresponding to X¹ to X³) that are carboxyl groups, or ester groups decomposable by biological enzyme reaction. In this case, R^{3′}, R^{4′} and R^{5′} do not exist.

That is, the hydroxycitric acid derivatives employable in the invention are compounds as described hereinabove in which at least one of the hydroxyl groups is modified while the carboxyl groups are unmodified, or in which at least one of the hydroxyl groups and at least one carboxyl group are modified. Specific examples of such compounds include compounds having combinations of the atoms and groups represented by R¹¹, R¹², X¹ to X³, R³, R⁴, R⁵, R^{3′}, R^{4′} and R^{5′}.

As described above, the functional groups in the molecule can be modified in various combinations. In particular, preferred examples include:

compounds of the formula (2-1) below in which R¹² is a hydrogen atom, R³ to R⁵ are all hydrogen atoms, and X¹ to X³ are all oxygen atoms (for example, hydroxycitric acid-2-palmitate):

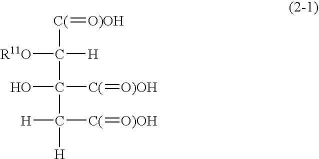

compounds of the formula (2-2) below in which R¹² is a hydrogen atom, R³ to R⁵ are each a hydrogen atom or a chain hydrocarbon group of 1 to 30 carbon atoms that may have a branch or an unsaturated bond (with the proviso that R³ to R⁵ cannot be hydrogen atoms at the same time), and X¹ to X³ are all oxygen atoms:

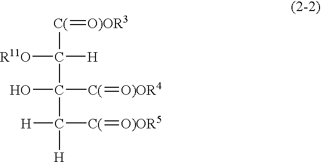

Specific examples of the hydroxycitric acid derivatives include hydroxycitric acid-2-octanoate, hydroxycitric acid-2-caprate, hydroxycitric acid-2-laurate, hydroxycitric acid-2-myrystate, hydroxycitric acid-2-palmitate, hydroxycitric acid-2-stearate, hydroxycitric acid-2-behenoate, hydroxycitric acid-2-isopalmitate, hydroxycitric acid-2-isostearate, hydroxycitric acid-2-hexyldecanoate, hydroxycitric acid-2-rinoletae, hydroxycitric acid monomethylester-2-myrystate, hydroxycitric acid monomethylester-2-palmitate, hydroxycitric acid monomethylester-2-stearate and salts thereof.

Of these, preferred are hydroxycitric acid-2-laurate, hydroxycitric acid-2-myrystate, hydroxycitric acid-2-palmitate, hydroxycitric acid-2-stearate, hydroxycitric acid-2-behenoate, hydroxycitric acid-2-isopalmitate, hydroxycitric acid-2-isostearate, hydroxycitric acid-2-hexyldecanoate, hydroxycitric acid-2-rinoletae and salts thereof.

More preferred are hydroxycitric acid-2-myrystate, hydroxycitric acid-2-palmitate, hydroxycitric acid-2-stearate and salts thereof.

The pharmacologically acceptable salts of the hydroxycitric acid derivatives include alkali metal salts and alkaline earth metal salts of the aforesaid hydroxycitric acid derivatives.

The alkali metal salts include sodium salts and potassium salts. The alkaline earth metal salts include calcium salts.

The hydroxycitric acid derivatives and salts thereof can be produced by any processes without limitation. For example, the hydroxycitric acid and/or alkali metal salt thereof and/or alkaline earth metal salt thereof may be reacted in an appropriate solvent with a carboxylic, phosphoric or sulfonic acid derivative breakable in the living body.

The hydroxycitric acid, derivatives thereof and pharmacologically acceptable salts thereof may be used singly or in combination of two or more kinds.

The invention desirably employs at least one of the hydroxycitric acid, derivatives thereof and pharmacologically acceptable salts thereof, in an amount of 0.01 to 20% by mass, and preferably 0.05 to 10% by mass of the slimming skin external preparation.

(α-2 Adrenergic Inhibitor)

Another example of the slimming ingredients are those capable of depressing functions of α-2 adrenaline that inhibits synthesis of intracellular cAMP which acts as a second messenger in lipolysis. Namely, because the α-2 adrenaline inhibits lipolysis, depressing the α-2 adrenaline will lead to a secondary effect of stimulation of lipolysis.

The α-2 adrenergic inhibitors include yohimbine, phentolamine, phenoxybenzamine, tolazoline, ergotamine, ergotoxine, dihydroergotamine, ergometrine, methylergometrine, dihydroergotoxine, rauwolscine, piperoxan, derivatives thereof and pharmacologically acceptable salts thereof. Examples further include natural products having the function of the α-2 adrenergic inhibitors, particularly plant extracts such as a ginkgo extract, a hedera rhombea extract and a chestnut extract. The pharmacologically acceptable salts include inorganic acid salts, for example hydrochlorides. These may be used singly or in combination of two or more kinds.

The α-2 adrenergic inhibitor desirably constitutes 0.0001 to 1:0% by mass, and preferably 0.0005 to 5% by mass of the slimming skin external preparation.

(β-Adrenergic Stimulant)

Another example of the slimming ingredients are β-adrenergic stimulants capable of stimulating the synthesis of intracellular cAMP which acts as a second messenger in lipolysis. The β-adrenergic stimulants are expected to inhibit fat accumulation.

The β-adrenergic stimulants include isoproterenol, epinephrine, norepinephrine, dobutamine, dopamine, butopamine, salbutamol, terbutaline, isoetharine, protokylol, fenoterol, metaproterenol, clorprenaline, hexoprenaline, trimetoquinol, procaterol hydrochloride, prenalterol, forskolin, disodium(R,R)-5-[2-[[2-(3-chlorophenyl)-2-hydroxyethyl]-amino]propyl]-1,3-benzodioxol-2,2-dicarboxylate, (R,R)-4-[2-({2-[(3-chlorophenyl)-2-hydroxyethyl]amino}propyl)phenyl]phenoxyacetic acid, {2-hydroxy-5-[2-({2-hydroxy-3-[4-(1-methyl-4-trifluoromethyl)-1H-imidazol-2-yl]phenoxy}propyl)amino]ethoxy}-benzamide monomethane sulfonate, erythro-DL-1-(7-methylindane-4-yloxy)-3-isopropylaminobutane-2-ol, derivatives thereof and pharmacologically acceptable salts thereof. Examples further include natural products having the function of the β-adrenergic stimulants, particularly plant extracts such as a coleus forskohlii extract (forskolin), an ipomoea hederacea extract, an ipomoea batata extract, a salvia officinalis extract, a salvia miltiorrhiza extract and a rosmarinus officinalis (rosemary) extract.

The pharmacologically acceptable salts include inorganic acid salts, for example hydrochlorides. These may be used singly or in combination of two or more kinds.

The β-adrenergic stimulant desirably constitutes 0.0001 to 10% by mass, and preferably 0.0005 to 5% by mass of the slimming skin external preparation.

(Inhibitor of Phosphodiesterase)

A further example of the slimming ingredients are inhibitors of phosphodiesterase having effects of the decomposition of intracellular cAMP which acts as a second messenger in lipolysis. The phosphodiesterase lowers the intercellular concentration of cAMP, weakens the activity of hormone-sensitive lipase, and inhibits lipolysis. Accordingly, depressing the phosphodiesterase will permit lipolysis and lead to a secondary effect of stimulation of lipolysis.

The inhibitors of phosphodiesterase include caffeine, theophylline, theobromine, aminophylline, xanthine, isobutylmethylxanthine, apigenin, amentoflavone, bilobetin, sciadopiticin, ginkgonetin, derivatives thereof and pharmacologically acceptable salts thereof. Examples further include natural products having the function of the inhibitors of phosphodiesterase, particularly plant extracts such as a tea extract, a coffee extract, a guarana extract, a yerba mate extract, a cola extract, a ginkgo extract, a sequoia sempervirens extract, a yew extract and a selaginella shakotanensis extract. The pharmacologically acceptable salts include alkali metal salts and alkaline earth metal salts. The alkali metal salts include sodium salts, and the alkaline earth salts include calcium salts. These may be used singly or in combination of two or more kinds.

The inhibitor of phosphodiesterase desirably constitutes 0.0001 to 10% by mass, and preferably 0.0005 to 5% by mass of the slimming skin external preparation.

Skin-Care Ingredients

The carnitine derivative functions to increase the in-tissue concentration of the carnitine which acts as a and pharmacologically acceptable salts thereof. Examples further include naturally occurring fatty acids such as coconut oil fatty acids. The pharmacologically acceptable salts include alkali metal salts such as sodium salts and potassium salts. These may be used singly or in combination of two or more kinds.

The saturated or unsaturated fatty acid of 12 to 22 carbon atoms that may have a branch, and/or the pharmacologically acceptable salt thereof desirably constitutes 0.001 to 20% by mass, and preferably 0.005 to 10% by mass of the slimming skin external preparation.

(Ascorbic Acids and Ascorbic Acid Derivatives)

Other examples of the skin-care ingredients are ascorbic acids, ascorbic acid derivatives, and pharmacologically acceptable salts thereof. The invention may employ one or more such skin-case ingredients. It is a known fact that they stimulate collagen synthesis and possess skin-care functions including an elimination function of active oxygen. Therefore, these skin-care ingredients are widely used in cosmetics.

The ascorbic acids include L-ascorbic acid. The pharmacologically acceptable salts of the ascorbic acids include sodium L-ascorbate and magnesium L-ascorbate.

The ascorbic acid derivatives include L-ascorbyl stearate, L-ascorbyl palmitate, L-ascorbyl dipalmitate, mediator when the fatty acid is incorporated into mitochondrion, and thereby enhances the fat metabolism. However, excessive fat metabolism causes shortage of subcutaneous fat and sebum to result in the lack of skin vitality, dry skin and skin roughness.

In view of this problem, combined use of the carnitine derivative and a particular skin-care ingredient provides a slimming skin external preparation capable of facilitating the subcutaneous lipolysis and imparted with a skin-care function to keep the skin healthy.

(Fatty Acids)

The skin-care ingredients include fatty acids.

Percutaneous administration of a preparation containing the carnitine derivative and fatty acids gives a slimming effect in the subcutaneous area by the carnitine derivative stimulating the lipolysis, and supplies the superficial skin with the appropriate nutrient fatty acids. Accordingly, good slimming effects are achieved while moisturizing the skin. The fatty acids include saturated or unsaturated fatty acids of 12 to 22 carbon atoms that may have a branch, and pharmacologically acceptable salts thereof. Specific examples include lauric acid, myristic acid, palmitic acid, stearic acid, behenic acid, oleic acid, isostearic acid, 12-hydroxystearic acid, undecylenic acid, hexyldecanoic acid L-ascorbyl tetraisopalmitate and L-ascorbic acid-2-glucoside. Examples further include ascorbic acid-2-phosphate and higher fatty acid esters thereof, represented by the following formula (4): 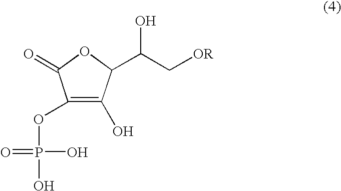
wherein R is a hydrogen atom or an acyl residue of the higher fatty acid.

The pharmacologically acceptable salts of the ascorbic acid derivatives include disodium L-ascorbate sulfate. Examples further include sodium salts, potassium salts, magnesium salts and zinc salts of the ascorbic acid-2-phosphate and higher fatty acid esters thereof, represented by the above formula (4).

These may be used singly or in combination of two or more kinds.

Of these, preferred are the higher fatty acid esters of the ascorbic acid-2-phosphate represented by the above formula (4), and sodium salts, potassium salts, magnesium salts and zinc salts thereof. More preferred are the higher fatty acid esters of the ascorbic acid-2-phosphate represented by the above formula (4) in which R is an acyl residue of lauric acid, myristic acid, palmitic acid, stearic acid or 2-hexyldecanoic acid, and sodium salts, potassium salts, magnesium salts and zinc salts thereof.

The invention desirably employs at least one of the ascorbic acids, ascorbic acid derivatives and pharmacologically acceptable salts thereof in an amount of 0.01 to 20% by mass, and preferably 0.05 to 10% by mass of the slimming skin external preparation.

(Tocopherols and Tocopherol Derivatives)

Another examples of the skin-care ingredients are tocopherols, tocopherol derivatives and pharmacologically acceptable salts thereof. The invention may employ one or more such skin-care ingredients. Tocopherols, known as vitamin E, possess effects of antioxidation, biomembrane stabilization, immunostimulation and blood-flow facilitation. Cosmetics imparted with such effects are known.

The tocopherols include α-tocopherol, β-tocopherol, γ-tocopherol and δ-tocopherol.

The tocopherol derivatives include α-tocopherol derivatives, β-tocopherol derivatives, γ-tocopherol derivatives and δ-tocopherol derivatives. Examples further include phosphates and aminoalkylcarboxylates thereof.

These may be used singly or in combination or two or more kinds.

The tocopherol phosphates are preferably represented by the following formula (5): 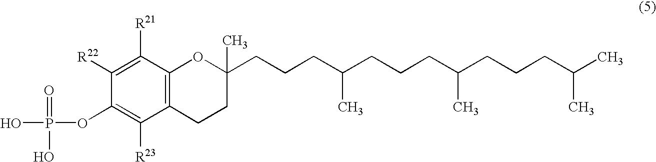
wherein R²¹, R²² and R²³ are each a hydrogen atom or a methyl group.

The tocopherol aminoalkylcarboxylates are preferably represented by the following formula (6): 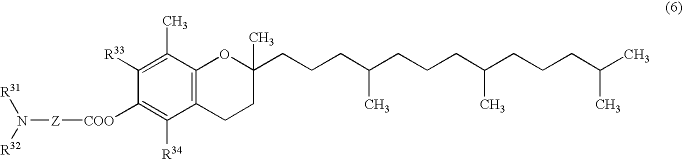
wherein R³¹ and R³² are the same or different lower alkyl groups or each a hydrogen atom, R³³ and R³⁴ are each a hydrogen atom or a methyl group, and Z is a branched or linear alkylene group that may have a substituent group.

Examples of the aminoalkylcarboxylic acids of the tocopherol aminoalkylcarboxylates include glycine, alanine, β-alanine, valine, leucine, isoleucine, phenylalanine, methionine, cysteine, serine, threonine, tyrosine, thyroxine, histidine, proline, 4-hydroxyproline, aspartic acid, glutamic acid, N-alkyl derivatives thereof and N,N-dialkyl derivatives thereof.

The pharmacologically acceptable salts include inorganic acid salts and alkali metal salts of the above tocopherols and tocopherol derivatives. The inorganic acid salts include hydrochlorides, and the alkali metal salts include sodium salts and potassium salts.

The invention desirably employs at least one of the tocopherols, tocopherol derivatives and pharmacologically acceptable salts thereof in an amount of 0.01 to 10% by mass, and preferably 0.05 to 5% by mass of the slimming skin external preparation.

Other Ingredients

The above-described slimming ingredients and/or skin-care ingredients may be used together with or may be replaced with other ingredients commonly used in skin external preparations and cosmetics, while still achieving the effects of the present invention. (If the ingredients listed below include any ingredients described above as the slimming or skin-care ingredients, such ingredients are understood as the slimming or skin-care ingredients.)

The compoundable ingredients are not particularly limited and include:

hydrocarbons such as ozokerite, α-olefin oligomers, light isoparaffin, light liquid isoparaffin, squalene, squalane, synthetic squalane, vegetable squalane, ceresin, paraffin, polyethylene powder, polybutene, microcrystalline wax, liquid isoparaffin, liquid paraffin, mineral oil and vaseline;

natural fats and oils, such as natural waxes including jojoba oil, carnaubawax, candelilla wax, ricebran wax, shellac, lanolin, mink oil wax, whale wax, sugarcane wax, sperm oil, beeswax and montan wax; avocado oil, almond oil, olive oil, extra virgin olive oil, sesame oil, rice bran oil, rice oil, rice germ oil, corn oil, soybean oil, maize oil, persic oil, palm kernel oil, palm oil, castor oil, grape seed oil, cotton seed oil, coconut oil, hydrogenated coconut oil, beef tallow, hardened oil, horse oil, mink oil, egg yolk oil, egg yolk fatty oil, rose hip oil, kukui nut oil, evening primrose oil, wheat germ oil, peanut oil, camellia oil, sasanqua oil, cacao butter, Japanese wax, beef bone fat, neatsfoot oil, lard, horse fat, mutton tallow, shea butter, macadamia nut oil and meadowfoam oil;

fatty acids such as lauric acid, myristic acid, palmitic acid, stearic acid, behenic acid, oleic acid, isostearic acid, 12-hydroxystearic acid, undecylenic acid and coconut fatty acid;

higher monoalcohols such as isostearyl alcohol, octyldodecanol, hexyldecanol, cholesterol, phytosterol, lauryl alcohol, myristyl alcohol, cetanol, stearyl alcohol, oleyl alcohol, behenyl alcohol and cetostearyl alcohol;

alkyl glyceryl ethers such as batyl alcohol, chimyl alcohol, selachyl alcohol and isostearyl glyceryl ether;

esters such as isopropyl myristate, butyl myristate, isopropyl palmitate, ethyl stearate, butyl stearate, ethyl oleate, ethyl linoleate, isopropyl linoleate, cetyl caprylate, hexyl laurate, isooctyl myristate, decyl myristate, myristyl myristate, cetyl myristate, octadecyl myristate, cetyl palmitate, stearyl stearate, decyl oleate, oleyl oleate, cetyl ricinoleate, isostearyl laurate, isotridecyl myristate, isocetyl myristate, isostearyl myristate, octyldodecyl myristate, 2-ethylhexyl palmitate, isocetyl palmitate, isostearyl palmitate, 2-ethylhexyl stearate, isocetyl stearate, isodecyl oleate, octyldodecyl oleate, octyldodecyl ricinoleate, ethyl isostearate, isopropyl isostearate, cetyl 2-ethylhexanoate, cetostearyl 2-ethylhexanoate, stearyl 2-ethylhexanoate, hexyl isostearate, ethylene glycol dioctanoate, ethylene glycol dioleate, propylene glycol dicaprylate, propylene glycol di(caprylate caprate), propylene glycol dicaprate, propylene glycol dioleate, neopentyl glycol dicaprate, neopentyl glycol dioctanoate, glyceryl tricaprylate, glyceryl tri-2-ethylhexanoate, glyceryl tri(caprylate caprate), glyceryl tri(caprylate caprate stearate), glyceryl triundecylate, glyceryl triisopalmitate, glyceryl triisostearate, trimethylolpropane tri-2-ethylhexanoate, trimethylolpropane triisostearate, pentaerythrityl tetra-2-ethylhexanoate, pentaerythrityl tetramyristate, pentaerythrityl tetraisostearate, diglyceryl tetraisostearate, octyldodecyl neopentanoate, isocetyl octanoate, isostearyl octanoate, 2-ethylhexyl isopelargonate, hexyldecyl dimethyloctanoate, octyldodecyl dimethyloctanoate, 2-ethylhexyl isopalmitate, isocetyl isostearate, isostearyl isostearate, octyldodecyl isostearate, lauryl lactate, myristyl lactate, cetyl lactate, octyldodecyl lactate, triethyl citrate, acetyltriethyl citrate, acetyltributyl citrate, trioctyl citrate, triisocetyl citrate, trioctyldodecyl citrate, diisostearyl malate, 2-ethylhexyl hydroxystearate, di-2-ethylhexyl succinate, diisopropyl adipate, diisobutyl adipate, dioctyl adipate, diheptylundecyl adipate, diethyl sebacate, diisopropyl sebacate, dioctyl sebacate, cholesteryl stearate, cholesteryl isostearate, cholesteryl hydroxystearate, cholesteryl oleate, dihydrocholesteryl oleate, phytosteryl isostearate, phytosteryl oleate, isocetyl 12-stearoylhydroxystearate, stearyl 12-stearoylhydroxystearate, isostearyl 12-stearoylhydroxystearate, polyoxyethylene (3) polyoxypropylene (1) cetyl ether acetate, polyoxyethylene (3) polyoxypropylene (1) isocetyl ether acetate, isononyl isononanoate, octyl isononanoate, tridecyl isononanoate and isotridecyl isononanoate; silicone oils such as methyl polysiloxane, methylphenyl polysiloxane, methylhydrogen polysiloxane, methyl cyclopolysiloxane, octamethyl cyclotetrasiloxane, decamethyl cyclopentasiloxane, dodecamethyl cyclohexasiloxane, octamethyl trisiloxane, decamethyl tetrasiloxane, tetradecamethyl hexasiloxane, highly polymerized methyl polysiloxane, dimethyl siloxane/methyl(polyoxyethylene)siloxane/methyl(polyoxypropylene)siloxane copolymer, dimethyl siloxane/methyl(polyoxyethylene) siloxane copolymer, dimethyl siloxane/methyl(polyoxypropylene)siloxane copolymer, dimethyl siloxane/methyl cetyloxysiloxane copolymer, dimethyl siloxane/methyl stearoxysiloxane copolymer, polyether-modified silicones, alcohol-modified silicones, alkyl-modified silicones and amino-modified silicones;

polymers such as sodium alginate, carrageenan, agar, furcelleran, cyamoposis gum, pyrus cyclonia seed, konjac mannan, tamarind gum, tara gum, dextrin, starch, locust bean gum, gum arabic, ghatti gum, karaya gum, tragacanth gum, arabinogalactan, pectin, marmelo, chitosan, starch, curdlan, xanthan gum, gellan gum, cyclodextrin, dextran, pullulan, microcrystalline cellulose, methylcellulose, ethylcellulose, hydroxyethylcellulose, hydroxypropylcellulose, hydroxypropylmethylcellulose, carboxymethylcellulose, carboxy starch, cationized cellulose, starch phosphate, cationized cyamoposis gum, carboxymethyl/hydroxypropylated cyamoposis gum, hydroxypropylated cyamoposis gum, albumin, casein, gelatin, sodium polyacrylate, polyacrylic acid amide, carboxyvinyl polymers, polyethyleneimine, highly polymerized polyethylene glycol, polyvinyl alcohol, polyvinylpyrrolidone, polyvinyl ether, polyacrylamide, acrylic acid copolymers, methacrylic acid copolymers, maleic acid copolymers, vinylpyridine copolymers, ethylene/acrylic acid copolymers, vinylpyrrolidone polymers, vinyl alcohol/vinylpyrrolidone copolymers, nitrogen-substituted acrylamide polymers, amino-modified silicones, cationized polymers, dimethylacryl ammonium polymers, acrylic acid-based anionic polymers, methacrylic acid-based anionic polymers, modified silicones, alkyl(C₁₀₋₃₀) acrylate or methacrylate copolymers and polyoxyethylene/polyoxypropylene copolymer;

lower monoalcohols such as ethanol, isopropyl alcohol, 1-butanol, 2-butanol and benzyl alcohol;

polyhydric alcohols such as ethylene glycol, diethylene glycol, polyethylene glycol, propylene glycol, polypropylene glycol, glycerol, diglycerol, polyglycerol, 1,3-butanediol, triethylene glycol, dipropylene glycol, 3-methyl-1,3-butanediol, 1,2-pentanediol, 1,4-pentanediol, 1,5-pentanediol, 2,4-pentanediol, 2-methyl-2,4-pentanediol, 3-methyl-1,5-pentanediol, 1,2-hexanediol and 1,6-hexanediol;

anionic surfactants such as coconut fatty acid potassium ester, coconut fatty acid sodium ester, coconut fatty acid triethanolamine ester, potassium laurate, sodium laurate, triethanolamine laurate, potassium myristate, sodium myristate, isopropanolamine myristate, potassium palmitate, sodium palmitate, isopropanolamine palmitate, potassium stearate, sodium stearate, triethanolamine stearate, potassium oleate, sodium oleate, castor oil fatty acid sodium ester, zinc undecylenate, zinc laurate, zinc myristate, magnesium myristate, zinc palmitate, zinc stearate, calcium stearate, magnesium stearate, aluminum stearate, calcium myristate, magnesium myristate, aluminum dimyristate, aluminum isostearate, polyoxyethylene laurylether acetic acid, sodium polyoxyethylene laurylether acetate, polyoxyethylene tridecylether acetic acid, sodium polyoxyethylene tridecylether acetate, sodium stearoyl lactate, sodium isostearoyl lactate, lauroylsarcosine sodium, coconut fatty acid sarcosine ester, coconut fatty acid sarcosine sodium ester, coconut fatty acid sarcosine triethanolamine ester, lauroyl sarcosine, lauroyl sarcosine potassium, lauroyl sarcosine triethanolamine, oleoyl sarcosine, myristoyl sarcosine sodium, sodium stearoyl glutamate, coconut fatty acid acylglutamic acid, coconut fatty acid potassium acylglutamate, coconut fatty acid sodium acylglutamate, coconut fatty acid triethanolamine acylglutamate, lauroyl acylglutamic acid, potassium lauroyl acylglutamate, sodium lauroyl acylglutamate, triethanolamine lauroyl acylglutamate, myristoyl acylglutamic acid, potassium myristoyl acylglutamate, sodium myristoyl acylglutamate, stearoyl acylglutamic acid, potassium stearoyl acylglutamate, disodium stearoyl acylglutamate, hardened tallow fatty acid sodium acylglutamate, coconut fatty acid/hardened tallow fatty acid sodium acylglutamate, coconut fatty acid methylalanine sodium ester, lauroyl methylalanine, lauroyl methylalanine sodium, lauroyl methylalanine triethanolamine, myristoyl methylalanine sodium, lauroyl methyltaurine sodium, coconut fatty acid methyltaurine potassium ester, coconut fatty acid methyltaurine sodium, coconut fatty acid methyltaurine magnesium ester, myristoyl methyltaurine sodium, palmitoyl methyltaurine sodium, stearoyl methyltaurine sodium, oleoyl methyltaurine sodium, sodium alkanesulfonate, sodium tetradecenesulfonate, dioctylsodium sulfosuccinate, lauryl disodium sulfosuccinate, coconut fatty acid ethyl ester sodium sulfonate, sodium laurylsulfate, triethanolamine laurylsulfate, sodium cetyl sulfate, triethanolamine alkylsulfates (11, 13, 15), sodium alkylsulfates (12, 13), triethanolamine alkylsulfates (12, 13), ammonium alkylsulfates (12, 14, 16), diethanolamine alkylsulfates (12, 13), triethanolamine alkylsulfates (12-14), triethanolamine alkylsulfates (12-15), magnesium triethanolamine cocoalkylsulfate, ammonium laurylsulfate, potassium laurylsulfate, magnesium laurylsulfate, monoethanolamine laurylsulfate, diethanolamine laurylsulfate, sodium myristylsulfate, sodium stearylsulfate, sodium oleylsulfate, triethanolamine oleylsulfate, sodium polyoxyethylene laurylether sulfate, triethanolamine polyoxyethylene laurylether sulfate, sodium polyoxyethylene (1) alkyl (11, 13, 15) ether sulfate, triethanolamine polyoxyethylene (1) alkyl (11, 13, 15) ether sulfate, sodium polyoxyethylene (3) alkyl (11-15) ether sulfate, sodium polyoxyethylene (2) alkyl (12, 13) ether sulfate, sodium polyoxyethylene (3) alkyl (12-14) ether sulfate, sodium polyoxyethylene (3) alkyl (12-15) ether sulfate, sodium polyoxyethylene (2) laurylether sulfate, sodium polyoxyethylene (3) myristylether sulfate, higher fatty acid alkanolamide sulfate sodium, laurylphosphoric acid, sodium laurylphosphate, potassium cetylphosphate, diethanolamine cetylphosphate, polyoxyethylene oleylether phosphoric acid, polyoxyethylene laurylether phosphoric acid, sodium polyoxyethylene laurylether phosphate, polyoxyethylene cetylether phosphoric acid, sodium polyoxyethylene cetylether phosphate, polyoxyethylene stearylether phosphoric acid, polyoxyethylene oleylether phosphoric acid, sodium polyoxyethylene oleylether phosphate, polyoxyethylene alkylphenyl ether phosphoric acid, sodium polyoxyethylene alkylphenyl ether phosphate, triethanolamine polyoxyethylene alkylphenyl ether phosphate, polyoxyethylene octylether phosphoric acid, polyoxyethylene (10) alkyl (12, 13) ether phosphoric acid, polyoxyethylene alkyl (12-15) ether phosphoric acid, polyoxyethylene alkyl (12-16) ether phosphoric acid, triethanolamine polyoxyethylene laurylether phosphate and diethanolamine polyoxyethylene oleylether phosphate;

cationic surfactants such as dioctylamine, dimethylstearylamine, trilaurylamine, stearic acid diethylaminoethylamide, lauryltrimethylammonium chloride, cetyltrimethylammonium chloride, cetyltrimethylammonium bromide, cetyltrimethylammonium saccharin, stearyltrimethylammonium chloride, alkyl (20-22) trimethylammonium chloride, lauryltrimethylammonium bromide, alkyl (16, 18) trimethylammonium chloride, stearyltrimethylammonium bromide, stearyltrimethylammonium saccharin, alkyl (28) trimethylammonium chloride, di(polyoxyethylene)oleylmethylammonium chloride (2EO), dipolyoxyethylenestearylmethylammonium chloride, polyoxyethylene (1) polyoxypropylene (25) diethylmethylammonium chloride, tri(polyoxyethylene)stearylammonium chloride (SEQ), distearyldimethylammonium chloride, dialkyl (12-15) dimethylammonium chloride, dialkyl (12-18) dimethylammonium chloride, dialkyl (14-18) dimethylammonium chloride, dicocoyldimethylammonium chloride, dicetyldimethylammonium chloride, isostearyllauryldimethylammonium chloride, benzalkonium chloride, myristyldimethylbenzylammonium chloride, lauryldimethyl(ethylbenzyl)ammonium chloride, stearyldimethylbenzylammonium chloride, laurylpyridinium chloride, cetylpyridinium chloride, lauroylcolaminoformylmethylpyridinium chloride, stearoylcolaminoformylmethylpyridinium chloride, alkylisoquinolium bromide, methylbenzethonium chloride and benzethonium chloride;

amphoteric surfactants such as 2-alkyl-N-carboxymethyl-N-hydroxyethyl imidazolinium betaine, alkyldiaminoethylglycine hydrochloride, lauryldiaminoethylglycine sodium, undecylhydroxyethylimidazolium betaine sodium, undecyl-N-carboxymethylimidazolium betaine, coconut fatty acid acyl-N-carboxyethyl-N-hydroxyethylethylenediamine disodium ester, coconut fatty acid acyl-N-carboxyethoxyethyl-N-carboxyethylethylenediamine disodium ester, coconut fatty acid acyl-N-carboxymethoxyethyl-N-carboxymethylethylenediamine disodium ester, sodium laurylaminopropionate, sodium laurylaminodipropionate, triethanolamine laurylaminopropionate, palm oil fatty acid acyl-N-carboxyethyl-N-hydroxyethylethylenediamine sodium ester, betaine lauryldimethylaminoacetate, coconut oil alkyldimethylaminoacetic acid betaine ester, betaine stearyldimethylaminoacetate, stearyldimethyl betaine sodium, coconut fatty acid amidopropylbetaine ester, palm oil fatty acid amidopropylbetaine ester, lauric acid amide betaine propylacetate, amidopropylbetaine ricinoleate, stearyldihydroxyethyl betaine and laurylhydroxysulfobetaine;

nonionic surfactants such as polyoxyethylene (10) alkyl (12, 13) ether, polyoxyethylene lauryl ether, polyoxyethylene cetyl ether, polyoxyethylene stearyl ether, polyoxyethylene oleyl ether, polyoxyethylene (3, 7, 12) alkyl (12-14) ether, polyoxyethylene tridecyl ether, polyoxyethylene myristyl ether, polyoxyethylene-sec-alkyl (14) ether, polyoxyethylene isocetyl ether, polyoxyethylene cetostearyl ether, polyoxyethylene (2, 10, 20) isostearyl ether, polyoxyethylene oleylcetyl ether, polyoxyethylene (20) aralkyl ether, polyoxyethylene octyldodecyl ether, polyoxyethylene behenyl ether, polyoxyethylene octylphenyl ether, polyoxyethylene nonylphenyl ether, polyoxyethylene dinonylphenyl ether, polyoxyethylene (1) polyoxypropylene (1, 2, 4, 8) cetyl ether, polyoxyethylene (5) polyoxypropylene (1, 2, 4, 8) cetyl ether, polyoxyethylene (10) polyoxypropylene (1, 2, 4, 8) cetyl ether, polyoxyethylene (20) polyoxypropylene (1, 2, 4, 8) cetyl ether, polyoxyethylene polyoxypropylene lauryl ether, polyoxyethylene (3) polyoxypropylene (34) stearyl ether, polyoxyethylene (4) polyoxypropylene (30) stearyl ether, polyoxyethylene (34) polyoxypropylene (23) stearyl ether, polyoxyethylene polyoxypropylene cetyl ether, polyoxyethylene polyoxypropylene decyltetradecyl ether, polyethylene glycol monolaurate, ethylene glycol monostearate, polyethylene glycol monostearate, polyethylene glycol monooleate, ethylene glycol fatty acid ester, self-emulsifiable ethylene glycol monostearate, diethylene glycol laurate, polyethylene glycol myristate, polyethylene glycol palmitate, diethylene glycol stearate, self-emulsifiable polyethylene glycol (2) monostearate, polyethylene glycol isostearate, ethylene glycol dioctanoate, diethylene glycol dilaurate, polyethylene glycol dilaurate, polyethylene glycol (150) dipalmitate, ethylene glycol distearate, diethylene glycol distearate, polyoxyethylene coconut oil alkyldimethylamine oxide;

natural surfactants such as saponin, lecithin, soybean phospholipid, hydrogenated soybean phospholipid, soybean lysophospholipid, hydrogenated soybean lysophospholipid, egg yolk lecithin, hydrogenated egg yolk lysophosphatidylcholine, phosphatidylcholine, phosphatidylethanolamine, phosphatidylserine, sphingophospholipid, sphingomyelin, ganglioside, bile acid, cholic acid, deoxycholic acid, sodium cholate, sodium deoxycholate, spiculisporic acid, rhamnolipid, trehalose lipid, sophoroliquid and mannosylerythritol lipid;

ultraviolet light absorbers, including paraminobenzoic acid derivatives such as paraminobenzoic acid, ethyl paraminobenzoate, glyceryl paraminobenzoate, amyl paradimethylaminobenzoate and 2-ethylhexyl paradimethylaminobenzoate, cinnamic acid derivatives such as benzyl cinnamate, diparamethoxy cinnamic acid glyceryl mono-2-ethylhexanoate, methyl 2,4-diisopropylcinnamate, ethyl 2,4-diisopropylcinnamate, potassium paramethoxycinnamate, sodium paramethoxycinnamate, isopropyl paramethoxycinnamate, 2-ethylhexyl paramethoxycinnanate, 2-ethoxyethyl paramethoxycinnamate and ethyl paraethoxycinnamate, urocanic acid derivatives such as urocanic acid and ethyl urocanate, benzophenone derivatives such as 2,4-dihydroxybenzophenone, polyethylene glycol distearate, ethylene glycol dioleate, polyethylene glycol dioleate, polyethylene glycol diricinoleate, polyoxyethylene (20) sorbitan monolaurate, polyoxyethylene (20) sorbitan monopalmitate, polyoxyethylene (6) sorbitan monostearate, polyoxyethylene (20) sorbitan monostearate, polyoxyethylene (20) sorbitan tristearate, polyoxyethylene (6) sorbitan monooleate, polyoxyethylene (20) sorbitan monooleate, polyoxyethylene (20) sorbitan trioleate, polyoxyethylene (20) coconut fatty acid sorbitan, polyoxyethylene (10-80) sorbitanmonolaurate, pblyoxyethylene sorbitan tristearate, polyoxyethylene (20) sorbitan isostearate, polyoxyethylene (150) sorbitan tristearate, polyoxyethylene castor oil, polyoxyethylene hardened castor oil, polyoxyethylene (10) hardened castor oil, polyoxyethylene (20) hardened castor oil, polyoxyethylene (40) hardened castor oil, polyoxyethylene (50) hardened castor oil, polyoxyethylene (60) hardened castor oil, lipophilic glyceryl monostearate, lipophilic glyceryl monooleate, self-emulsifiable glyceryl monostearate, coconut fatty acid glyceryl, glyceryl laurate, glyceryl myristate, glyceryl isostearate, glyceryl ricinoleate, glyceryl monohydroxystearate, glyceryl oleate, glyceryl linoleate, glyceryl erucate, glyceryl behenate, wheat germ oil fatty acid glyceride, safflower oil fatty acid glyceryl, hydrogenated soybean fatty acid glyceryl, saturated fatty acid glyceride, cotton seed oil fatty acid glyceryl, monoisostearic acid glyceryl monomyristate, monotallow fatty acid glyceride, monoglyceryl lanolin fatty acid, glyceryl sesquioleate, glyceryl distearate, glyceryl diisostearate, glyceryl diarachidate, sorbitan monolaurate, sorbitan monopalmitate, sorbitan monostearate, sorbitan monoisostearate, sorbitan monooleate, sorbitan sesquistearate, sorbitan sesquioleate, sorbitan tristearate, sorbitan trioleate, coconut fatty acid sorbitan, sorbitan isostearate, sorbitan sesquiisostearate, sorbitan distearate, diglyceryl isopalmitate, poly (4-10) glyceryl monolaurate, poly (10) glyceryl monomyristate, poly (2-10) glyceryl monostearate, poly (2-10) glyceryl monoisostearate, poly (2-10) glyceryl monooleate, diglyceryl sesquioleate, poly (2-10) glyceryl diisostearate, poly (6-10) glyceryl distearate, diglyceryl triisostearate, poly (10) glyceryl tristearate, poly (10) glyceryl trioleate, poly (2) glyceryl tetraisostearate, decaglyceryl pentastearate, poly (6-10) glyceryl pentaoleate, poly (10) glyceryl heptastearate, decaglyceryl decastearate, poly (10) glyceryl decaoleate, condensed poly (6) glyceryl ricinoleate, cane sugar fatty acid ester, cane sugar coconut fatty acid ester, alkyl glucoside, coconut oil alkyldimethylamine oxide, lauryldimethylamine oxide, dihydroxyethyllauryldimethylamine oxide, stearyldimethylamine oxide, oleyldimethylamine oxide and 2,2′,4,4′-tetrahydroxybenzophenone, 2-hydroxy-4-methoxy-5-sulfobenzophenonesodium, 2-hydroxy-4-methoxybenzophenone-5-sulfonic acid, 2-hydroxy-4-methoxybenzophenone, 2,2′-dihydroxy-4,4′-dimethoxybenzophenone and 2,2′-dihydroxy-4,4′-dimethoxy-5-sulfobenzophenonesodium, salicylic acid derivatives such as ethylene glycol salicylate, 2-ethylhexyl salicylate, phenyl salicylate, benzyl salicylate, p-tert-butylphenyl salicylate, homomethyl salicylate and 3,3,5-trimethylcyclohexyl salicylate, 2-(2′-hydroxy-5′-methoxyphenyl)benzotriazole and 4-tert-butyl-4′-methoxybenzoylmethane;

powders and color materials such as kaolin, silicic anhydride, aluminum magnesium silicate, sericite, talc, boron nitride, mica, montmorillonite, hemp cellulose powder, wheat starch, silk powder, cornstarch, natural dyes including nitro dye, azo dye, nitroso dye, triphenylmethane dye, xanthene dye, quinoline dye, anthraquinone dye, indigo dye, pyrene dye, phthalocyanine dye, flavonoid, quinone, porphyrin, water-soluble annatto, squidinkpowder, caramel, guaiazulene, gardenia blue, gardenia yellow, cochineal, shikonin, copper chlorophyllin sodium, paprika dye, safflower red, safflower yellow, laccaic acid and riboflavin butyrate, carbon black, yellow iron oxide, black iron oxide, red iron oxide, iron blue, ultramarine blue, zinc oxide, chromium oxide, titanium oxide, black titanium oxide, zirconium oxide, chromium hydroxide, alumina, magnesium oxide, barium sulfate, aluminum hydroxide, calcium carbonate, lithium cobalt titanate, manganese violet and pearl pigment;

plant extracts such as angelica keiskei extract, gambir extract, avocado extract, hydrangea serrata leaf extract, gynostemma pentaphyllum extract, althea extract, arnica extract, oil-soluble arnica extract, almond extract, aloe extract, styrax benzoin resin extract, ginkgo extract, urtica extract, orris root extract, fennel extract, curcuma extract, rose fruit extract, echinacea leaf extract, scutellaria baicalensis root extract, phellodendron bark extract, coptis rhizome extract, hordeum vulgare seed extract, gumbo extract, hypericum erectum extract, oil-soluble hypericum erectum extract, lamium album flower extract, oil-soluble lamium album flower extract, ononis extract, nasturtium officinale extract, orange flower water, kaki tannin, puerariae radix extract, valerian extract, cattail extract, chamomilla extract, oil-soluble chamomilla extract, chamomilla water, oat extract, carrot extract, oil-soluble carrot extract, carrot oil, artemisia capillaris extract, licorice extract, licorice extract powder, licorice flavonoid, cantharis tincture, raspberry extract, kiwi extract, cinchona bark extract, cucumber extract, apricot kernel extract, quince seed extract, gardenia extract, sasa veitchii extract, sophora angustifolia extract, walnut shell extract, clematis extract, brown sugar extract, chlorella extract, mulberry extract, cinnamon bark extract, gentian extract, geranium herb extract, tea extract, spatterdock extract, arctium lappa root extract, oil-soluble arctium lappa root extract, wheat germ extract, hydrolyzed wheat powder, rice bran extract, rice bran fermentation extract, comfrey extract, asiasarum root extract, saffron extract, saponaria officinalis extract, oil-soluble salvia extract, crataegus cuneata fruit extract, xanthoxylum extract, shiitake mushroom extract, shiitake mushroom extract powder, rehmannia glutinosa extract, sycon extract, oil-soluble sycon extract, Japanese basil extract, linden extract, oil-soluble linden extract, filipendula multijuga extract, crude drug extract, coix lacryma-jobi seed extract, ginger extract, oil-soluble ginger extract, ginger tincture, acorus calamus root extract, betula alba extract, oil-soluble betula alba extract, betula alba sap, lonicera extract, equisetum arvense extract, oil-soluble equisetum arvense extract, scordinin, stevia extract, ivy extract, crataegus oxyacantha extract, sambucus nigra flower extract, juniperus communis extract, achillea millefolium extract, oil-soluble achillea millefolium extract, mentha piperita extract, sage extract, oil-soluble sage extract, sage water, malva sylvestris extract, celery extract, cnidium officinale extract, cnidium officinale water, swertia japonica extract, soybean extract, jujube extract, thyme extract, camellia sinensis leaf extract, camellia sinensis dry distillate, camellia sinensis seed extract, clove flower extract, citrus unshiu peel extract, camellia japonica seed extract, centella asiatica extract, oil-soluble juglans regia extract, duke extract, terminalia extract, angelica acutiloba extract, oil-soluble angelica acutiloba extract, angelica acutiloba water, calendula officinalis flower extract, oil-soluble calendula officinalis flower extract, soymilk powder, prunus persica extract, citrus aurantium amara extract, houttuynia cordata extract, tomato extract, potentilla erecta root extract, natto extract, ginseng extract, oil-soluble ginseng extract, garlic extract, rosa canina fruit extract, oil-soluble rosa canina fruit extract, malt extract, malt root extract, ophiopogon tuber extract, parsley extract, hordeum vulgare leaf juice concentrate, distilled peppermint water, hamamelis water, hamamelis extract, rosa centifolia flower extract, parietaria extract, isodonis japonicus extract, eriobotrya japonica leaf extract, oil-soluble eriobotrya japonica leaf extract, coltsfoot flower extract, poria cocos extract, ruscus aculeatus root extract, ruscus aculeatus root extract powder, grape extract, grape leaf extract, grape water, hayflower extract, luffa cylindrica fruit extract, luffa cylindrica fruit water, safflower extract, oil-soluble tilia miqueliana extract, tilia miqueliana water, paeonia suffruticosa root extract, hops extract, oil-soluble hops extract, pinus sylvestris cone extract, silybum marianum fruit extract, horse chestnut extract, oil-soluble horse chestnut extract, sapindus mukurossi peel extract, melissa officinalis leaf extract, melilotus officinalis extract, peach leaf extract, oil-soluble peach leaf extract, bean-sprouts extract, centaurea cyanus flower extract, centaurea cyanus flower water, eucalyptus extract, saxifraga sarmentosa extract, lilium candidum bulb extract, coix lacryma jobi seed extract, oil-soluble coix lacryma jobi seed extract, artemisia princeps extract, artemisia princeps water, lavender extract, lavender water, apple extract, ganoderma lucidum extract, lettuce extract, astragalus sinicus extract, rose water, rosemary extract, oil-soluble rosemary extract, anthemis nobilis flower extract and sanguisorba officinalis root extract;

amino acids and peptides such as glycine, valine, leucine, isoleucine, serine, threonine, phenylalanine, tyrosine, tryptophan, cystine, cysteine, methionine, hydroxyproline, aspartic acid, asparagine, glutamic acid, glutamine, histidine, γ-aminobutyric acid, DL-pyrrolidonecarboxylic acid, ε-aminocaproic acid, hydrolyzed elastin, water-soluble elastin, hydrolyzed collagen, water-soluble collagen, casein, glutathione, wheat peptide and soybean peptide;

vitamins and vitamin affecters, including vitamin A such as retinol, retinal, retinoic acid, retinol acetate and retinol palmitate, carotenoids such as α-carotene, β-carotene, γ-carotene, δ-carotene, lycopene, zeaxanthin, cryptoxanthin, echinenone and astaxanthin, vitamin B1 such as thiamines, vitamin B2 such as riboflavin, vitamin B6 such as pyridoxine, pyridoxal and pyridoxamine, vitamin B12 such as cyanocobalamin, vitamin C such as folic acids, nicotinic acid, nicotinic acid amide, pantothenic acids, biotins, L-ascorbic acid, sodium L-ascorbate, L-ascorbyl stearate, L-ascorbyl palmitate, L-ascorbyl dipalmitate, L-ascorbyl tetraisopalmitate, disodium L-ascorbate sulfate, magnesium L-ascorbate, L-ascorbyl sodium phosphate, ascorbic acid-2-phosphoric acid and L-ascorbic acid-2-glucoside, vitamin D such as ergocalciferol and cholecalciferol, vitamin E such as d-α-tocopherol, DL-α-tocopherol, dl-α-tocopherol acetate, dl-α-tocopherol succinate, β-tocopherol, γ-tocopherol and d-δ-tocopherol, ubiquinones, vitamin K, carnitine, ferulic acid, γ-oryzanol, α-lipoic acid and orotic acid;

antiseptics such as benzoic acid, sodium benzoate, undecylenic acid, salicylic acid, sorbic acid, potassium sorbate, dehydroacetic acid, sodium dehydroacetate, isobutyl paraoxybenzoate, isopropyl paraoxybenzoate, ethyl paraoxybenzoate, butyl paraoxybenzoate, propyl paraoxybenzoate, benzyl paraoxybenzoate, methyl paraoxybenzoate, methyl sodium paraoxybenzoate, phenoxyethanol, photosensitive agent No. 101, photosensitive agent No. 201 and photosensitive agent No. 401;

antioxidants such as butylhydroxyanisole, butylhydroxytoluene, propyl gallate, erythorbic acid, sodium erythorbate, parahydroxyanisole and octyl gallate;

sequestering agents such as metal-ionic compound including trisodium ethylenediaminehydroxyethyltriacetate, edetic acid, disodium edetate, trisodium edetate, tetrasodium edetate, sodium citrate, gluconic acid, phytic acid, sodium polyphosphate and sodium metaphosphate;

moisturizers such as hyaluronic acid, sodium hyaluronate, sodium chondroitinsulfate, sodium lactate, sodium pyrrolidonecarboxylate, betaine, lactic acid bacteria culture solution, yeast extract and ceramide;

antiinflammatory agents such as glycyrrhizinic acid, trisodium glycyrrhizinate, dipotassium glycyrrhizinate, monoammonium glycyrrhizinate, β-glycyrrhetinic acid, glycerol glycyrrhetinate, stearyl glycyrrhetinate, lysozyme chloride, hydrocortisone and allantoin;

pH adjusters such as sodium hydroxide, potassium hydroxide and triethanolamine;

salts such as sodium chloride, potassium chloride, magnesium chloride and sodium sulfate;

α-hydroxy acids such as citric acid, glycolic acid, tartaric acid and lactic acid;

whitening agents such as arbutin, α-arbutin and placental extract;

essential oils such as angelica oil, ylang ylang oil, elemi oil, German chamomile oil, anthemis nobilis oil, cardamom oil, calamus oil, galbanum oil, camphor oil, carrot seed oil, clary sage oil, clove oil, cinnamon bark oil, coriander oil, cypress oil, sandalwood oil, cedarwood oil, citronella oil, cinnamon leaf oil, jasmine absolute, juniper berry oil, ginger extract, spearmint oil, sage oil, cedar oil, geranium oil, thyme oil, tea tree oil, nutmeg oil, niaouli oil, neroli oil, pine oil, basil oil, peppermint oil, patchouli oil, palmarosa oil, fennel oil, petitgrain oil, black pepper oil, frankincense oil, vetivert oil, peppermint oil, bergamot oil, benzoin oil, aniba rosaeodora oil, marjoram oil, myrrh oil, melissa oil, eucalyptus oil, ravensara oil, lavandin oil, lavender oil, lindane oil, rose oil, rosewood oil, rosemary oil and lovage oil;

terpenes such as pinene, terpinene, terpinolene, myrcene and longifolene; perfumes and water.

The slimming skin external preparation and cosmetic containing the external preparation may be in any forms or formulations that are used in contact with skin. Preferably, the form and formulation are such that the external preparation or cosmetic can be used in contact with the skin including the area where the metabolism of subcutaneous fat is desired. In the broad sense, the formulations include skim milks, skin creams, foundation creams, massage creams, cleansing creams, shaving creams, cleansing foams, skin toners, lotions, packs, lipsticks, rouges, eye shadows, manicures, soaps, body shampoos, hand soaps, shampoos, conditioners, hair tonics, treatment conditioners, hair creams, hair sprays, hair growth tonics, baldness remedies, hairdyes, styling spritz, depilatories, antidandruff agents, toothpastes, denture adhesives, mouthwashes, permanent wave agents, curling agents, styling agents, ointments, adhesive skin patches, taping agents, bath agents, antiperspirants and sunscreen agents. Although the formulations are not particularly limited as long as they are contacted with skin when used, cosmetics are a particularly preferable formulation. The external preparation and cosmetic can be used regardless of user's gender and age, and can be used for animal skin as well as human skin.

The slimming skin external preparation and cosmetic of the invention may be in any states such as solid, liquid, semisolid and gas, and may be in any forms including powder, granules, tablets, gels and foams, although not particularly limited thereto.

The cosmetic according to the present invention may contain ingredients of the aforementioned additional ingredients that are commonly used in cosmetics. In addition, existing cosmetic ingredients may be used. For example, any of the cosmetic ingredients listed in the following documents are employable: The Japanese Standards of Cosmetic Ingredients 2nd edition (edited by Society of Japanese Pharmacopoeia and published by Yakuji Nippo, Ltd. (1984)), The Japanese Cosmetic Ingredients Codex (edited by Ministry of Health and Welfare, Pharmaceutical Examination Division and published by Yakuji Nippo, Ltd. (1993)), Supplement to The Japanese Cosmetic Ingredients Codex (edited by Ministry of Health and Welfare, Pharmaceutical Examination Division and published by Yakuji Nippo, Ltd. (1993)), The Comprehensive Licensing Standards of Cosmetics by Category (edited by Ministry of Health and Welfare, Pharmaceutical Examination Division and published by Yakuji Nippo, Ltd. (1993)), The Japanese Cosmetic Ingredients Codex by Category (edited by Ministry of Health and Welfare, Pharmaceutical Examination Division and published by Yakuji Nippo, Ltd. (1997)), Dictionary of Cosmetic Ingredients (Nikko Chemicals., Co. Ltd. (1991)), and Latest Cosmetic Functional Materials 300 (CMS Publishing Co., Ltd. (2002)).

The slimming skin external preparation and cosmetic of the present invention can be produced by common methods depending on the formulations, for example by dissolving, mixing or dispersing the aforesaid ingredients in predetermined amounts.

### EXAMPLES

Hereinbelow, the present invention will be described in greater detail by examples. However, it should be construed that the invention is not limited thereto.

### Synthesis Example 1

### Synthesis of hexadecanoyl-L-carnitine hydrochloride

A 500-ml four-necked flask equipped with a dropping funnel, a thermometer and a cooling tube was charged with L-carnitine (1 mol) and trifluoroacetic acid (350 ml), followed by heating at 60° C. with stirring to obtain a solution. To the resultant uniform reaction liquid, hexadecanoyl chloride (1.1 mol) was added dropwise from the dropping funnel over a period of 30 minutes. After completion of the dropwise addition, the reaction liquid was stirred at 60° C. for 2 hours. Thereafter, the trifluoroacetic acid was evaporated using an evaporator. The residue was dissolved in n-hexane (500 ml), then combined with water (500 ml), and stirred for 30 minutes. The liquid mixture was combined with ethanol (500 ml) and methyl tert-butyl ether (500 ml) to perform extraction. The aqueous phase was collected and was combined with n-butanol (500 ml) and further with water (100 ml) to perform extraction. The n-butanol phase was separated and the solvent was evaporated to afford 201 g of a residue. The residue was purified by recrystallization in isopropanol to give 99% pure hexadecanoyl-L-carnitine hydrochloride (0.3 mol).

### Synthesis Example 2

### Synthesis of hexanoyl-L-carnitine hydrochloride, tetradecanoyl-L-carnitine hydrochloride and octadecanoyl-L-carnitine hydrochloride

Hexanoyl-L-carnitine hydrochloride, tetradecanoyl-L-carnitine hydrochloride and octadecanoyl-L-carnitine hydrochloride, each having 99% purity, were obtained in the same manner as in Synthesis Example 1 except that hexadecanoyl chloride was replaced with equimolar amounts of hexanoyl chloride, tetradecanoyl chloride and octadecanoyl chloride.

### Synthesis Example 3

### Synthesis of Hydroxycitric Acid-2-Palmitate

### (1) Synthesis of hydroxycitric acid tribenzyl ester

A 200-ml evaporation flask was charged with 2.96 g (10.1 mmol) of calcium hydroxycitrate, 5.86 g (30.8 mmol) of toluenesulfonic acid monoanhydride, 10 g (92.5 mmol) of benzyl alcohol and 20 ml of toluene. These were stirred under reflux for 4 hours with azeotropic water removal. After cooled naturally, the mixture was combined with 50 ml of ethyl acetate, and these were stirred well. The resultant mixture in small portions was introduced with stirring into a 500-ml beaker containing 100 ml of a 5% by mass aqueous solution of sodium hydrogencarbonate. The insolubles were removed, the aqueous phase was separated, and the organic phase washed with water and was dried over anhydrous sodium sulfate. The solvent and benzyl alcohol were removed by vacuum evaporation, and the residue was analyzed by silica gel column chromatography. Elution using a 5:1 mixture of hexane and ethyl acetate gave 1.96 g of the objective compound as a white solid (40% yield)

### (2) Synthesis of hydroxycitric acid tribenzyl ester-2-palmitate

A 50-ml evaporation flask was charged with 239 mg (0.50 mmol) of hydroxycitric acid tribenzyl ester synthesized in (1), 5 ml of tetrahydrofuran (THF) and 165 mg (0.60 mmol) of palmitic acid chloride. The flask cooled with ice was further charged with a solution of 61 mg (0.60 mmol) of triethylamine in 2 ml of THF. Stirring was performed for 30 minutes at the temperature and for 2 hours at room temperature. To the reaction liquid, 100 ml and 50 ml of ethyl acetate and water respectively were added. The organic phase washed in the usual way and was dried over anhydrous sodium sulfate. The solvent was removed by vacuum evaporation, and the residue was analyzed by silica gel column chromatography. Elution using a 10:1 mixture of hexane and ethyl acetate gave 330 mg of the objective compound as a white solid (92% yield).

### (3) Synthesis of hydroxycitric acid-2-palmitate

A 50-ml evaporation flask was charged with 300 mg (0.42 mmol) of hydroxycitric acid tribenzyl ester-2-palmitate synthesized in (2), 5 ml of ethanol and 5 ml of dimethylformamide (DMF). Subsequently, 40 mg of 10% by mass-palladium activated carbon was added as a catalyst, and catalytic reduction was carried out for 2 hours. The catalyst was filtered off, and the solvent was removed by vacuum evaporation. To the residue, hexane was added. The solid precipitated was filtered and 175 mg of the objective compound was obtained as a white solid (84% yield).

The compound structure was identified by the ¹H-NMR spectrum given below:

¹H-NMR (270 MHz, DMSO-D6, ppm): 5.0 (s, 1H, CH), 3.2-3.8 (br, 4H, OH, COOH), 2.7-3.0 (dd, 2H, —CH₂COOH), 2.0-2.2 (m, 2H, —CH₂COOC—), 1.0-1.5 (m, 26H, —(CH₂)₁₃—), 0.8-0.9 (t, 3H, CH₃—)

### Synthesis Example 4

### Synthesis of dl-α-tocopherol-N,N-dimethylglycine ester hydrochloride

59.1 g and 40.0 g of dicyclohexylcarbodiimide and N,N-dimethylglycine hydrochloride respectively were dissolved in 320 g of pyridine. The solution was combined with a solution of 60.0 g of dl-α-tocopherol in 240 g of pyridine. Reaction was performed at room temperature for 8 hours with stirring, and the pyridine was removed by evaporation. The residue was combined with 2000 ml of water and 1000 ml of ethyl acetate, and further with approximately 40 g of sodium carbonate to adjust the pH in the range of 7 to 8. The ethyl acetate phase was separated, and the aqueous phase was extracted three times, each with 200 ml of ethyl acetate. The ethyl acetate was evaporated. Thereafter, ethyl acetate was newly added and the mixture was dried over anhydrous sodium sulfate. The solid was filtered and was conditioned such that the concentration of dl-α-tocopherol dimethylglycine ester would be in the range of 7 to 8%. Subsequently, a 20%-hydrochloric acid dioxane solution was added in a molar amount 1.5 times in terms of hydrochloric acid that of the dl-α-tocopherol dimethylglycine ester to achieve neutrality. The thus-formed solid was filtered and was subjected to recrystallization using a methanol/acetone solvent. Thus, 49.2 g of dl-α-tocopherol-N,N-dimethylglycine ester hydrochloride was obtained.

### Example 1

(Comparison of Percutaneous Absorption Properties)

The following five carnitine and carnitine derivatives were tested:

1) L-Carnitine (standard reference)

2) Hexanoyl-L-carnitine hydrochloride (Synthetic Example 2)

3) Tetradecanoyl-L-carnitine hydrochloride (Synthetic Example 2)

4) Hexadecanoyl-L-carnitine hydrochloride (Synthetic Example 1)

5) Octadecanoyl-L-carnitine hydrochloride (Synthetic Example 2)

The test substances were each dissolved in Dulbecco's PBS (−) to give a 1% by mass solution of the test substance. The test substance solutions were placed, each 50 μL, to a tissue surface of human skin tissue three dimensional model (TESTSKIN™ LSD-d, available from TOYOBO CO., LTD.), and were incubated at 37° C. in a 5% CO₂ atmosphere for 2 hours. Thereafter the test substance solutions were aspirated out, and the skin models were further incubated at 37° C. in a 5% CO₂ atmosphere for 2, 6 or 12 hours. The skin models were sampled after the lapse of each of the incubation times. The skin models sampled at the incubation times were washed with Dulbecco's PBS (−), and the tissue surfaces on which the test substances had been placed were punched out with a punch 6 mm in diameter. The skin models were then homogenated in a HEPES buffer solution (pH: 7.2), and were quantitatively analyzed using a high performance liquid chromatography to determine the carnitine content in the skin model. (Measurement conditions were: column: Shodex DE-413 (available from Showa Denko K.K.), temperature: 25° C., eluting solutions: acetonitrile/0.1 M H₃PO₄=67/33, flow rate: 1.0 ml/min, detector: differential refractometer detector.) Separately, the protein content in the skin models was quantitatively determined by the Lowry method.

From the analysis results, the carnitine contents in the skin models (unit: nmol/mg skin protein) were obtained for the test substances at each of the incubation times. The results are shown in Table 1.

TABLE 1

| | After 2-hour | After 6-hour | After 12-hour |
|---|---|---|---|
| Test substance | incubation | incubation | incubation |
| 1) | 12 | 12 | 11 |
| 2) | 7 | 14 | 21 |
| 3) | 6 | 12 | 22 |
| 4) | 6 | 10 | 23 |
| 5) | 5 | 10 | 25 |

The above results show that the administration of the acylated carnitine derivatives can increase the in-tissue concentration of carnitine more effectively than the administration of L-carnitine, from immediately after the administration over a long term.

### Example 2

(Measurement of Fat Metabolic Activity for Mouse 3T3 Adipocytes)

The five carnitine and carnitine derivatives 1) to 5) used in Example 1 were tested. The fat metabolic activity was measured and compared by quantitative determination of glycerol in the culture medium based on the fact that metabolism of the fat accumulated in adipocytes releases glycerol and free fatty acid to the culture medium.

Mouse 3T3 cells as the preadipocytes were disseminated on a 10 cm-diameter dish such that 1×10⁴ cells were on the dish. The cells were incubated at 37° C. in a 5% CO₂ atmosphere over a period of 1 week using a DMEM culture medium (available from Invitrogen) containing 10% FCS. Thereafter, the culture medium was replaced with a 10% FCS-containing DMEM culture medium that contained 0.25 M of dexamethasone, 0.5 mM of isobutylmethylxanthine and 10 μg/ml of insulin, and incubation was continued for another 2 days. Subsequently, the culture medium was replaced again with a 10% FCS-containing ordinary DMEM culture medium, and incubation was performed for another week, resulting in differentiation of the preadipocytes into adipocytes. Next, the culture medium was replaced with an ordinary culture medium that contained 250 μM of the carnitine or carnitine derivative of the five substances 1) to 5) used in Example 1, and the culture medium was recovered after 24 hours. The cells were washed with Dulbecco's PBS (−) and were recovered. Cells incubated in the absence of the test substances were used as the control. The glycerol content in the culture medium was quantitatively determined using Glycerol Test E Wako (Wako Pure Chemical Industries, Ltd.).

Table 2 shows the free glycerol contents provided by the test substances relative to the control (100%).

TABLE 2

| | Free glycerol content |
|---|---|
| Test substance | (% relative to the control) |
| Control (none) | 100 |
| 1) | 120 |
| 2) | 155 |
| 3) | 168 |
| 4) | 180 |
| 5) | 176 |

The above results show that the administration of the test substances results in increased amounts of free glycerol than by the control. In particular, the administration of the acylated carnitine derivatives provides higher contents of free glycerol, indicating enhanced fat metabolism.

### Example 3

(Subcutaneous Fat Breakdown Test)

The five carnitine and carnitine derivatives 1) to 5) used in Example 1 were tested.

A male Wister rat, 10 weeks old, was shaved on the abdomen, and the skin was removed together with subcutaneous fat tissues and was placed in a Franz diffusion cell. An upper cell on the skin surface was filled with a buffer solution containing the Example 1 test substance, and a lower cell was filled with Dulbecco's PBS (−). The diffusion cell was held at 37° C. and was allowed to stand for 8 or 16 hours. After the lapse of each of the standing times, PBS (−) was withdrawn from the lower cell. The content of free glycerol resulting from the fat breakdown was measured with use of Glycerol Test E Wako. The results are shown in Table 3. The results are averages of three measurements.

TABLE 3

Free glycerol (μmol/ml)

| Test substance | After 8 hours | After 16 hours |
|---|---|---|
| Control (none) | 20 | 20 |
| 1) | 120 | 125 |
| 2) | 116 | 138 |
| 3) | 112 | 146 |
| 4) | 125 | 170 |
| 5) | 117 | 158 |

The above results show that the administration of the test substances results in increased amounts of free glycerol. In particular, the administration of the acylated carnitine derivatives provides a continuous effect of breakdown stimulation.

### Example 4

(Comparison of Percutaneous Absorption Properties)

The carnitine contents in the skin model (unit: nmol/mg skin protein) were determined in the same manner as described in Example 1, except that the following two carnitine and carnitine derivative were tested:

1) L-Carnitine (standard reference)

6) L-Carnitine hexadecyl ester hydrochloride

The results are shown in Table 4.

TABLE 4

| | After 2-hour | After 6-hour | After 12-hour |
|---|---|---|---|
| Test substance | incubation | incubation | incubation |
| 1) | 12 | 12 | 11 |
| 6) | 5 | 11 | 26 |

The above results show that the administration of the esterified carnitine derivative increases the in-tissue concentration of carnitine more effectively over a long term than the administration of L-carnitine.

### Example 5

(Measurement of Fat Metabolic Activity for Mouse 3T3 Adipocytes)

The fat metabolic activity for mouse 3T3 adipocytes was determined based on the free glycerol content in the same manner as in Example 2, except that the two carnitine and carnitine derivative 1) and 6) used in Example 4 were tested. Table 5 shows the free glycerol contents provided by the test substances relative to the control (100%).

TABLE 5

| | Free glycerol content |
|---|---|
| Test substance | (% relative to the control) |
| Control (none) | 100 |
| 1) | 120 |
| 6) | 165 |

test substances results in increased amounts of free glycerol than by the control. In particular, the administration of the esterified carnitine derivative provides a higher content of free glycerol, indicating enhanced fat metabolism.

### Example 6

(Subcutaneous Fat Breakdown Test)

The free glycerol contents were measured in the same manner as in Example 3, except that the two carnitine and carnitine derivative 1) and 6) used in Example 4 were tested. The results are shown in Table 6. The results are averages of three measurements.

TABLE 6

Free glycerol (μmol/ml)

| Test substance | After 8 hours | After 16 hours |
|---|---|---|
| Control (none) | 20 | 20 |
| 1) | 120 | 125 |
| 6) | 110 | 116 |

The above results show that the administration of the test substances results in increased amounts of free glycerol. In particular, the administration of the esterified carnitine derivative provides a continuous effect of lipolysis stimulation.

### Example 7

(Comparison of Percutaneous Absorption Properties)

The carnitine contents in the skin model (unit: nmol/mg skin protein) were determined in the same manner as described in Example 1, except that the following three carnitine and carnitine derivatives were tested:

1) L-Carnitine (standard reference)

7) Hexadecanoyl-L-carnitine methyl ester hydrochloride

8) Hexadecanoyl-L-carnitine hexadecyl ester hydrochloride

The results are shown in Table 7.

TABLE 7

| | After 2-hour | After 6-hour | After 12-hour |
|---|---|---|---|
| Test substance | incubation | incubation | incubation |
| 1) | 12 | 12 | 11 |
| 7) | 2 | 9 | 19 |
| 8) | 2 | 8 | 21 |

The above results show that the administration of the acylated and esterified carnitine derivatives can increase the in-tissue concentration more effectively than the administration of L-carnitine, from immediately after the administration over a long term.

### Example 8

(Measurement of Fat Metabolic Activity for Mouse 3T3 Adipocytes)

The fat metabolic activity for mouse 3T3 adipocytes was determined based on the free glycerol content in the same manner as in Example 2, except that the three carnitine and carnitine derivatives 1), 7) and 8) used in Example 7 were tested. Table 8 shows the free glycerol contents provided by the test substances relative to the control (100%).

TABLE 8

| | Free glycerol content |
|---|---|
| Test substance | (% relative to the control) |
| Control (none) | 100 |
| 1) | 120 |
| 7) | 143 |
| 8) | 148 |

The above results show that the administration of the test substances results in increased amounts of free glycerol than by the control. In particular, the administration of the acylated and esterified carnitine derivatives provides higher contents of free glycerol, indicating enhanced fat metabolism.

### Example 9

(Subcutaneous Fat Breakdown Test)

The free glycerol contents were measured in the same manner as in Example 3, except that the three carnitine and carnitine derivatives 1), 7) and 8) used in Example 7 were tested. The results are shown in Table 9. The results are averages of three measurements.

TABLE 9

Free glycerol (μmol/ml)

| Test substance | After 8 hours | After 16 hours |
|---|---|---|
| Control (none) | 20 | 20 |
| 1) | 120 | 125 |
| 7) | 68 | 145 |
| 8) | 75 | 150 |

The above results show that the administration of the test substances results in increased amounts of free glycerol. In particular, the administration of the acylated and esterified carnitine derivatives provides a continuous effect of lipolysis stimulation.

### Example 10

(Compounding Effect of Acylcarnitine with Slimming Ingredient)

Mouse 3T3-F442A cells (available from DAINIPPON PHARMACEUTICAL CO., LTD.) were disseminated on a 96-well micro plate and were incubated at 37° C. in a 5% CO₂ atmosphere over a period of 2 weeks using a DMEM culture medium (available from Invitrogen) containing 10% by mass FCS.

After differentiation of most cells into adipocytes had been observed with a microscope, the culture medium was replaced with one that contained base acylcarnitine (hexadecanoyl-L-carnitine hydrochloride or octadecanoyl-L-carnitine hydrochloride) and 50 μM each of test substances shown in Table 10 below (garcinia extract contained 70% by weight of hydroxycitric acid and was added in terms of the hydroxycitric acid concentration, and ginkgo biloba extract was added at 30 μg/ml). The incubation was continued for another 4 days. The control was incubated using a culture medium that contained the base alone (no test substances).

After the 4-days incubation, the cells were washed three times with Dulbecco's PBS (−). The triglyceride amount accumulated in the cells was quantitatively determined by oil red staining. Table 10 shows the accumulated triglyceride amounts provided by the test substances relative to the control (100%).

TABLE 10

| | | | Base | |
|---|---|---|---|---|
| | | | Hexadecanoyl- | Octadecanoyl- |
| | | | L-carnitine | L-carnitine |
| | | | hydrochloride | hydrochloride |
| Test | None (control) | | (100) | (100) |
| substance | L-carnitine hydrochloride | | 99 | 102 |
| | Fat synthetic pathway | Calcium hydroxycitrate | 89 | 88 |
| | inhibitor | Hydroxycitric acid-2-palmitate | 70 | 72 |
| | | Garcinia extract | 90 | 93 |
| | α-2 Adrenergic | Phentolamine hydrochloride | 68 | 65 |
| | inhibitor | Ginkgo biloba extract | 72 | 76 |
| | β-Adrenergic | Isoproterenol hydrochloride | 60 | 64 |
| | stimulant | | | |
| | Inhibitor of | 3-Isobutyl-1-methylxanthine | 50 | 57 |
| | phosphodiesterase | Theophylline | 62 | 58 |

Base
Hexadecanoyl-L-carnitine hydrochloride (Synthesis Example 1)
Octadecanoyl-L-carnitine hydrochloride (Synthesis Example 2)
Test Substances
(Fat Synthetic Pathway Inhibitors)
Calcium hydroxycitrate (Sigma-Aldrich)
Hydroxycitric acid-2-palmitate (Synthesis Example 3)
Garcinia extract (Sabinsa Corporation)
(α-2 Adrenergic inhibitors)
Phentolamine hydrochloride (Sigma-Aldrich)
Ginkgo biloba extract (TOKIWA PHYTOCHEMICAL CO., LTD.)
(β-Adrenergic stimulants)
Isoproterenol hydrochloride (Sigma-Aldrich)
(Inhibitors of Phosphodiesterase)
3-Isobutyl-1-methylxanthine (Sigma-Aldrich)
Theophylline (Sigma-Aldrich)
(Others)
L-carnitine hydrochloride (Sigma-Aldrich)

The results shown in Table 10 establish that the combined use of acylcarnitine with particular slimming ingredients, namely, hydroxycitric acid derivatives, α-2 adrenergic inhibitors, β-adrenergic stimulants and inhibitors of phosphodiesterase, permits a specific high effect of fat reduction as compared with the use of acylcarnitine alone or together with carnitine.

### Example 11

Slimming skin external preparations were prepared in accordance with the compositions given in Table 11. (The same compounds as used in Example 10 were purchased from the same suppliers.) The preparations were stable in one-month storage at 25° C. and had properties suitable for external application.

TABLE 11

| | Sample No. | | | | | | | | | |
|---|---|---|---|---|---|---|---|---|---|---|
| | 1 | 2 | 3 | 4 | 5 | 6 | 7 | 8 | 9 | 10 |
| Sodium chloride | 0.2 | 0.2 | 0.2 | 0.2 | 0.2 | 0.2 | 0.2 | 0.2 | 0.2 | 0.2 |
| Tetrasodium EDTA | 0.2 | 0.2 | 0.2 | 0.2 | 0.2 | 0.2 | 0.2 | 0.2 | 0.2 | 0.2 |
| 1,2-hexanediol | 4.0 | 4.0 | 4.0 | 4.0 | 4.0 | 4.0 | 4.0 | 4.0 | 4.0 | 4.0 |
| Dipotassium glycyrrhizate | 0.1 | 0.1 | 0.1 | 0.1 | 0.1 | 0.1 | 0.1 | 0.1 | 0.1 | 0.1 |
| 1% aqueous solution of | 4.0 | 4.0 | 4.0 | 4.0 | 4.0 | 4.0 | 4.0 | 4.0 | 4.0 | 4.0 |
| sodium hyaluronate | | | | | | | | | | |
| Hexadecanoyl-L-carnitine | 0.3 | 0.3 | 0.3 | 0.3 | 0.3 | — | — | — | — | — |
| hydrochloride | | | | | | | | | | |
| Octadecanoyl-L-carnitine | — | — | — | — | — | 0.3 | 0.3 | 0.3 | 0.3 | 0.3 |
| hydrochloride | | | | | | | | | | |
| Hydroxycitric | 0.03 | — | — | — | — | 0.03 | — | — | — | — |
| acid-2-palmitate | | | | | | | | | | |
| Garcinia extract | — | 1.0 | — | — | — | — | 1.0 | — | — | — |
| Forskolin | — | — | 0.03 | — | — | — | — | 0.03 | — | — |
| Ginkgo biloba extract | — | — | — | 1.0 | — | — | — | — | 1.0 | — |
| Xanthine | — | — | — | — | 0.3 | — | — | — | — | 0.3 |
| Purified water | Rmdr. | Rmdr. | Rmdr. | Rmdr. | Rmdr. | Rmdr. | Rmdr. | Rmdr. | Rmdr. | Rmdr. |

Note:
The numbers are mass percentages relative to the total (100% by mass).
Forskolin was purchased from Sabinsa Corporation.
Xanthine was purchased from Sigma-Aldrich.

### Example 12

(Compounding of Skin-Care Ingredients and its Influence on Acylcarnitine Slimming Functions)

Mouse 3T3-F442A cells were incubated as described in Example 10, and differentiation into adipocytes was observed. Thereafter, the culture medium was replaced with one that contained base acylcarnitine (hexadecanoyl-L-carnitine hydrochloride or octadecanoyl-L-carnitine hydrochloride) and 50 μM each of test substances shown in Table 12 below. The incubation was continued for another 4 days. The control was incubated using a culture medium that contained the base alone (no test substances).

After the 4-days incubation, the cells were washed three times with Dulbecco's PBS (−). The triglyceride amount accumulated in the cells was quantitatively determined by oil red staining. Table 12 shows the accumulated triglyceride amounts provided by the test substances relative to the control (100%).

TABLE 12

| | | | Base | |
|---|---|---|---|---|
| | | | Hexadecanoyl- | Octadecanoyl- |
| | | | L-carnitine | L-carnitine |
| | | | hydrochloride | hydrochloride |
| Test | None (control) | | (100) | (100) |
| substance | Glycerol | | 118 | 113 |
| | Fatty acid | Myristic acid | 99 | 98 |
| | | Palmitic acid | 102 | 100 |
| | | Stearic acid | 101 | 99 |
| | Ascorbic acid | Ascorbic acid-2-phosphate magnesium | 98 | 97 |
| | derivative | salt | | |
| | | Ascorbic acid-2-phosphoric | 96 | 96 |
| | | acid-6-palmitate sodium salt | | |
| | | Ascorbic acid-2-glucoside | 99 | 102 |
| | Tocopherol | dl-α-Tocopherol phosphate sodium salt | 100 | 102 |
| | derivative | dl-α-Tocopherol-N,N-dimethylglycine | 98 | 99 |
| | | ester hydrochloride | | |

Base
Hexadecanoyl-L-carnitine hydrochloride (Synthesis Example 1)
Octadecanoyl-L-carnitine hydrochloride (Synthesis Example 2)
Test Substances
(Fatty acids)
Myristic acid (Sigma-Aldrich)
Palmitic acid (Sigma-Aldrich)
Stearic acid (Sigma-Aldrich)
(Ascorbic Acid Derivatives)
Ascorbic acid-2-phosphate magnesium salt (SHOWA DENKO K.K.)
Ascorbic acid-2-phosphoric acid-6-palmitate sodium salt (SHOWA DENKO K.K.)
Ascorbic acid-2-glucoside (Wako Pure Chemical Industries, Ltd.)
(Tocopherol Derivatives)
dl-α-Tocopherol phosphate sodium salt (Sigma-Aldrich)
dl-α-Tocopherol-N,N-dimethylglycine ester hydrochloride (Synthesis Example 4)
(Others)
Glycerol (Sigma-Aldrich)

Table 12 shows that the combination of acylcarnitine with glycerol results in rather high fat accumulation. This means that the glycerol compounded in the preparation possibly decreases the slimming function of acylcarnitine. On the other hand, the combined use of acylcarnitine and particular skin-care ingredients, namely, fatty acids, ascorbic acids and tocopherol derivatives, hardly affects the fat accumulation. That is, these skin-care ingredients are specific ingredients that can be compounded together with the acylcarnitine without deteriorating the acylcarnitine's slimming function.

### Example 13

Slimming skin external preparations were prepared in accordance with the compositions given in Table 13. The preparations were stable in three-month storage at 25° C. without any separation, sedimentation or remarkable turbidity and had properties suitable for external application.

TABLE 13

| Sample No. | 11 | 12 | 13 | 14 | 15 | 16 | 17 | 18 | 19 | 20 | 21 | 22 |
|---|---|---|---|---|---|---|---|---|---|---|---|---|
| Sodium chloride | 0.2 | 0.2 | 0.2 | 0.2 | 0.2 | 0.2 | 0.2 | 0.2 | 0.2 | 0.2 | 0.2 | 0.2 |
| Tetrasodium EDTA | 0.2 | 0.2 | 0.2 | 0.2 | 0.2 | 0.2 | 0.2 | 0.2 | 0.2 | 0.2 | 0.2 | 0.2 |
| 1,2-hexandiol | 4.0 | 4.0 | 4.0 | 4.0 | 4.0 | 4.0 | 4.0 | 4.0 | 4.0 | 4.0 | 4.0 | 4.0 |
| Dipotassium glycyrrhizate | 0.1 | 0.1 | 0.1 | 0.1 | 0.1 | 0.1 | 0.1 | 0.1 | 0.1 | 0.1 | 0.1 | 0.1 |
| 1% aqueous solution of | 4.0 | 4.0 | 4.0 | 4.0 | 4.0 | 4.0 | 4.0 | 4.0 | 4.0 | 4.0 | 4.0 | 4.0 |
| sodium hyaluronate | | | | | | | | | | | | |
| Hexadecanoyl-L-carnitine | 0.3 | 0.3 | 0.3 | 0.3 | 0.3 | 0.3 | — | — | — | — | — | — |
| hydrochloride | | | | | | | | | | | | |
| Octadecanoyl-L-carnitine | — | — | — | — | — | — | 0.3 | 0.3 | 0.3 | 0.3 | 0.3 | 0.3 |
| Palmitic acid | 0.03 | — | — | — | — | — | 0.03 | — | — | — | — | — |
| Stearic acid | — | 0.03 | — | — | — | — | — | 0.03 | — | — | — | — |
| Ascorbic acid-2-phosphate | — | — | 1.0 | — | — | — | — | — | 1.0 | — | — | — |
| Ascorbic acid-2-phosphoric | — | — | — | 1.0 | — | — | — | — | — | 1.0 | — | — |
| acid-6-palmitate sodium | | | | | | | | | | | | |
| salt | | | | | | | | | | | | |
| dl-α-Tocopherol phosphate | — | — | — | — | 0.3 | — | — | — | — | — | 0.3 | — |
| sodium salt | | | | | | | | | | | | |
| dl-α-Tocopherol-N,N- | — | — | — | — | — | 0.3 | — | — | — | — | — | 0.3 |
| dimethylglycine ester | | | | | | | | | | | | |
| hydrochloride | | | | | | | | | | | | |
| Purified water | Rmdr. | Rmdr. | Rmdr. | Rmdr. | Rmdr. | Rmdr. | Rmdr. | Rmdr. | Rmdr. | Rmdr. | Rmdr. | Rmdr. |

1. A slimming skin external preparation comprising a carnitine derivative represented by the following formula (1): 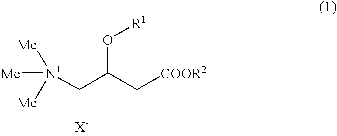
wherein R¹ is a hydrogen atom or an acyl group of 2 to 30 carbon atoms that may have a branch or an unsaturated bond, R² is a hydrogen atom or a hydrocarbon group of 1 to 22 carbon atoms that may have a branch or an unsaturated bond, R¹ and R² cannot be hydrogen atoms at the same time, Me is a methyl group, and X⁻ is an inorganic or organic anion that maintains electrical neutrality with a cation part of the carnitine derivative.

2. The slimming skin external preparation according to claim 1, wherein R¹ in the formula (1) is an acyl group of 4 to 22 carbon atoms that may have a branch or an unsaturated bond.

3. The slimming skin external preparation according to claim 1, wherein R¹ in the formula (1) is an acyl group of 14 to 22 carbon atoms that may have a branch or an unsaturated bond.

4. The slimming skin external preparation according to claim 1, wherein R² in the formula (1) is a hydrogen atom or a hydrocarbon group of 1 to 18 carbon atoms that may have a branch or an unsaturated bond.

5. The slimming skin external preparation according to claim 1, wherein R¹ in the formula (1) is a hydrogen atom and R² is a hydrocarbon group of 1 to 18 carbon atoms that may have a branch or an unsaturated bond.

6. The slimming skin external preparation according to claim 1, wherein R² in the formula (1) is a hydrocarbon group of 4 to 10 carbon atoms that may have a branch or an unsaturated bond.

7. The slimming skin external preparation according to claim 1, wherein R¹ in the formula (1) is an acyl group of 4 to 22 carbon atoms that may have a branch or an unsaturated bond and R² is a hydrogen atom.

8. The slimming skin external preparation according to claim 1, wherein the preparation contains the carnitine derivative in an amount of 0.01 to 20% by mass.

9. The slimming skin external preparation according to claim 1, wherein the preparation further contains at least one of hydroxycitric acid, a hydroxycitric acid derivative and a pharmacologically acceptable salt thereof.

10. The slimming skin external preparation according to claim 9, wherein the hydroxycitric acid is obtained from garcinia cambogia extract.

11. The slimming skin external preparation according to claim 9, wherein the hydroxycitric acid derivative is represented by the following formula (2): 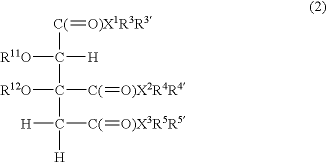
wherein R¹¹ and R¹² are each a hydrogen atom or a group detachable by biological enzyme reaction, the detachable group being represented by any of the following formula (3), R¹¹ and R¹² cannot be hydrogen atoms at the same time, X¹ to X³ are each a nitrogen atom or an oxygen atom, and R³, R⁴, R⁵, R^{3′}, R^{4′} and R^{5′} are each a hydrogen atom or a chain hydrocarbon group of 1 to 30 carbon atoms that may have a branch or an unsaturated bond (with the proviso that when X¹, X² or X³ is an oxygen atom, corresponding R^{3′}, R^{4′} or R^{5′} does not exist): 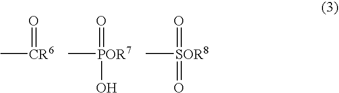
wherein R⁶ to R⁸ are each a hydrogen atom, an aryl group or a chain hydrocarbon group of 1 to 30 carbon atoms that may have a branch, an unsaturated bond or a substituent group.

12. The slimming skin external preparation according to claim 11, wherein R⁶ in the formula (3) is a chain hydrocarbon group of 7 to 23 carbon atoms that may have a branch, an unsaturated bond or a substituent group, and R⁷ and R⁸ are each a hydrogen atom or a chain hydrocarbon group of 8 to 24 carbon atoms that may have a branch, an unsaturated bond or a substituent group.

13. The slimming skin external preparation according to claim 11, wherein R³ to R⁵ in the formula (2) are each a hydrogen atom or a chain hydrocarbon group of 1 to 30 carbon atoms that may have a branch or an unsaturated bond, and X¹ to X³ are all oxygen atoms, and wherein R⁶ in the formula (3) is a chain hydrocarbon group of 7 to 23 carbon atoms that may have a branch, an unsaturated bond or a substituent group, and R⁷ and R⁸ are each a hydrogen atom or a chain hydrocarbon group of 8 to 24 carbon atoms that may have a branch, an unsaturated bond or a substituent group.

14. The slimming skin external preparation according to claim 11, wherein R¹² in the formula (2) is a hydrogen atom, R³ to R⁵ are all hydrogen atoms, and X¹ to X³ are all oxygen atoms, and wherein R⁶ in the formula (3) is a chain hydrocarbon group of 13 to 21 carbon atoms that may have a branch, an unsaturated bond or a substituent group.

15. The slimming skin external preparation according to claim 11, wherein R¹² in the formula (2) is a hydrogen atom, R³ to R⁵ are each a hydrogen atom or a chain hydrocarbon group of 1 to 30 carbon atoms that may have a branch or an unsaturated bond, R³ to R⁵ cannot be hydrogen atoms at the same time, and X¹ to X³ are all oxygen atoms, and wherein R⁶ in the formula (3) is a chain hydrocarbon group of 13 to 21 carbon atoms that may have a branch, an unsaturated bond or a substituent group.

16. The slimming skin external preparation according to claim 9, wherein the preparation contains at least one of the hydroxycitric acid, the hydroxycitric acid derivative and the pharmacologically acceptable salt thereof in an amount of 0.01 to 20% by mass.

17. The slimming skin external preparation according to claim 1, wherein the preparation further contains an α-2 adrenergic inhibitor.

18. The slimming skin external preparation according to claim 17, wherein the α-2 adrenergic inhibitor is at least one of yohimbine, phentolamine, phenoxybenzamine, tolazoline, ergotamine, ergotoxine, dihydroergotamine, ergometrine, methylergometrine, dihydroergotoxine, rauwolscine, piperoxan, derivatives thereof and pharmacologically acceptable salts thereof.

19. The slimming skin external preparation according to claim 17, wherein the α-2 adrenergic inhibitor is obtained from a plant extract.

20. The slimming skin external preparation according to claim 17, wherein the α-2 adrenergic inhibitor is obtained from at least one of a ginkgo extract, a hedera rhombea extract and a chestnut extract.

21. The slimming skin external preparation according to claim 1, wherein the preparation contains the α-2 adrenergic inhibitor in an amount of 0.0001 to 10% by mass.

22. The slimming skin external preparation according to claim 1, wherein the preparation further contains a β-adrenergic stimulant.

23. The slimming skin external preparation according to claim 22, wherein the β-adrenergic stimulant is at least one of isoproterenol, epinephrine, norepinephrine, dobutamine, dopamine, butopamine, salbutamol, terbutaline, isoetharine, protokylol, fenoterol, metaproterenol, clorprenaline, hexoprenaline, trimetoquinol, procaterol hydrochloride, prenalterol, forskolin, disodium(R,R)-5-[2-[[2-(3-chlorophenyl)-2-hydroxyethyl]-amino]propyl]-1,3-benzodioxol-2,2-dicarboxylate, (R,R)-4-[2-({2-[(3-chlorophenyl)-2-hydroxyethyl]amino}propyl)phenyl]phenoxyacetic acid, {2-hydroxy-5-[2-({2-hydroxy-3-[4-(1-methyl-4-trifluoromethyl)-1H-imidazol-2-yl]phenoxy}propyl)amino]ethoxy}-benzamide monomethane sulfonate, erythro-DL-1-(7-methylindane-4-yloxy)-3-isopropylaminobutane-2-ol, derivatives thereof and pharmacologically acceptable salts thereof.

24. The slimming skin external preparation according to claim 22, wherein the β-adrenergic stimulant is obtained from a plant extract.

25. The slimming skin external preparation according to claim 22, wherein the β-adrenergic stimulant is obtained from at least one of a coleus forskohlii extract (forskolin), an ipomoea hederacea extract, an ipomoea batata extract, a salvia officinalis extract, a salvia miltiorrhiza extract and a rosmarinus officinalis (rosemary) extract.

26. The slimming skin external preparation according to claim 22, wherein the preparation contains the β-adrenergic stimulant in an amount of 0.0001 to 10% by mass.

27. The slimming skin external preparation according to claim 1, wherein the preparation further contains an inhibitor of phosphodiesterase.

28. The slimming skin external preparation according to claim 27, wherein the inhibitor of phosphodiesterase is at least one of theophylline, theobromine, aminophylline, xanthine, isobutylmethylxanthine, apigenin, amentoflavone, bilobetin, sciadopiticin, ginkgonetin, derivatives thereof and pharmacologically acceptable salts thereof.

29. The slimming skin external preparation according to claim 27, wherein the inhibitor of phosphodiesterase is obtained from a plant extract.

30. The slimming skin external preparation according to claim 27, wherein the inhibitor of phosphodiesterase is obtained from at least one of a tea extract, a coffee extract, a guarana extract, a yerba mate extract, a cola extract, a ginkgo extract, a sequoia sempervirens extract, a yew extract and a selaginella shakotanensis extract.

31. The slimming skin external preparation according to claim 27, wherein the preparation contains the inhibitor of phosphodiesterase in an amount of 0.0001 to 10% by mass.

32. The slimming skin external preparation according to claim 1, wherein the preparation further contains a saturated or unsaturated fatty acid of 12 to 22 carbon atoms that may have a branch, and/or a pharmacologically acceptable salt thereof.

33. The slimming skin external preparation according to claim 32, wherein the fatty acid is at least one of lauric acid, myristic acid, palmitic acid, stearic acid, behenic acid, oleic acid, isostearic acid, 12-hydroxystearic acid, undecylenic acid and hexyldecanoic acid.

34. The slimming skin external preparation according to claim 32, wherein the fatty acid is a coconut oil fatty acid.

35. The slimming skin external preparation according to claim 32, wherein the preparation contains the saturated or unsaturated fatty acid of 12 to 22 carbon atoms that may have a branch, and/or the pharmacologically acceptable salt thereof in an amount of 0.001 to 20% by mass.

36. The slimming skin external preparation according to claim 1, wherein the preparation further contains at least one of ascorbic acid, an ascorbic acid derivative, and a pharmacologically acceptable salt thereof.

37. The slimming skin external preparation according to claim 36, wherein the ascorbic acid is L-ascorbic acid.

38. The slimming skin external preparation according to claim 36, wherein the pharmacologically acceptable salt of the ascorbic acid is sodium L-ascorbate and/or magnesium L-ascorbate.

39. The slimming skin external preparation according to claim 36, wherein the ascorbic acid derivative is at least one of L-ascorbyl stearate, L-ascorbyl palmitate, L-ascorbyl dipalmitate, L-ascorbyl tetraisopalmitate and L-ascorbic acid-2-glucoside.

40. The slimming skin external preparation according to claim 36, wherein the pharmacologically acceptable salt of the ascorbic acid derivative is disodium L-ascorbate sulfate.

41. The slimming skin external preparation according to claim 36, wherein the ascorbic acid derivative is ascorbic acid-2-phosphate and/or a higher fatty acid ester thereof, represented by the following formula (4): 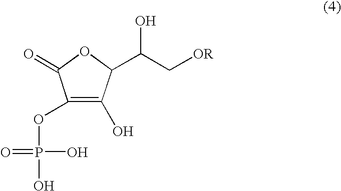
wherein R is a hydrogen atom or an acyl residue of the higher fatty acid.

42. The slimming skin external preparation according to claim 41, wherein R in the formula (4) is an acyl residue of lauric acid, myristic acid, palmitic acid, stearic acid or 2-hexyldecanoic acid.

43. The slimming skin external preparation according to claim 36, wherein the pharmacologically acceptable salt of the ascorbic acid derivative is a sodium salt, a potassium salt, a magnesium salt or a zinc salt of ascorbic acid-2-phosphate and/or a higher fatty acid ester thereof, represented by the following formula (4): 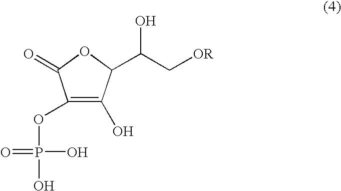
wherein R is a hydrogen atom or an acyl residue of the higher fatty acid.

44. The slimming skin external preparation according to claim 43, wherein R in the formula (4) is an acyl residue of lauric acid, myristic acid, palmitic acid, stearic acid or 2-hexyldecanoic acid.

45. The slimming skin external preparation according to claim 36, wherein the preparation contains at least one of the ascorbic acid, the ascorbic acid derivative and the pharmacologically acceptable salt thereof in an amount of 0.01 to 20% by mass.

46. The slimming skin external preparation according to claim 1, wherein the preparation further contains at least one of tocopherol, a tocopherol derivative and a pharmacologically acceptable salt thereof.

47. The slimming skin external preparation according to claim 46, wherein the tocopherol is at least one of α-tocopherol, β-tocopherol, γ-tocopherol and δ-tocopherol.

48. The slimming skin external preparation according to claim 46, wherein the tocopherol derivative is at least one of an α-tocopherol derivative, a β-tocopherol derivative, a γ-tocopherol derivative and a δ-tocopherol derivative.

49. The slimming skin external preparation according to claim 48, wherein the tocopherol derivative is tocopherol phosphate.

50. The slimming skin external preparation according to claim 49, wherein the tocopherol phosphate is a compound represented by the following formula (5): 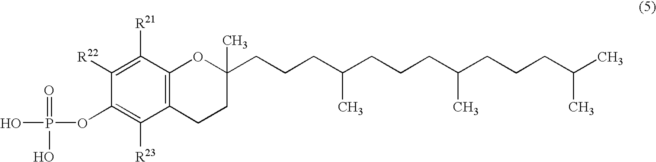
wherein R²¹, R²² and R²³ are each a hydrogen atom or a methyl group.

51. The slimming skin external preparation according to claim 48, wherein the tocopherol derivative is a tocopherol aminoalkylcarboxylate.

52. The slimming skin external preparation according to claim 51, wherein the tocopherol aminoalkylcarboxylate is a compound represented by the following formula (6): 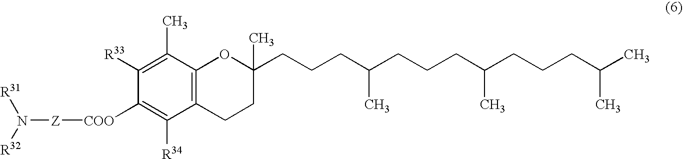
wherein R³¹ and R³² are the same or different lower alkyl groups or each a hydrogen atom, R³³ and R³⁴ are each a hydrogen atom or a methyl group, and Z is a branched or linear alkylene group that may have a substituent group.

53. The slimming skin external preparation according to claim 51, wherein the aminoalkylcarboxylic acid of the tocopherol aminoalkylcarboxylate is a compound selected from glycine, alanine, β-alanine, valine, leucine, isoleucine, phenylalanine, methionine, cysteine, serine, threonine, tyrosine, thyroxine, histidine, proline, 4-hydroxyproline, aspartic acid, glutamic acid, N-alkyl derivatives thereof and N,N-dialkyl derivatives thereof.

54. The slimming skin external preparation according to claim 46, wherein the preparation contains at least one of the tocopherol, the tocopherol derivative and the pharmacologically acceptable salt thereof in an amount of 0.01 to 10% by mass.

55. A cosmetic comprising the slimming skin external preparation of claim 1.

56. A cosmetic comprising the slimming skin external preparation of claim 9.

57. A cosmetic comprising the slimming skin external preparation of claim 17.

58. A cosmetic comprising the slimming skin external preparation of claim 22.

59. A cosmetic comprising the slimming skin external preparation of claim 27.

60. A cosmetic comprising the slimming skin external preparation of claim 32.

61. A cosmetic comprising the slimming skin external preparation of claim 36.

62. A cosmetic comprising the slimming skin external preparation of claim 46.
